SEQUENCE LISTING (11 sequences)

SEQ ID NO: 1 - DNA, 696 bp, Escherichia coli
atgtcaaacacgccaatcgagcttaaaggcagtagcttcactttatctgtggttcatctgcatgaggcagaacctaaggttatccatcaggcgctggaagacaaaatcgctcaggcccccgcatttttaaaacatgcccccgttgtactcaacgtcagtgcactggaagacccggtaaactggtcagcgatgcataaggcggtttcggcaaccggtttgcgggttattggcgtaagtggctgcaaagatgcgcaacttaaagccgaaattgaaaagatggggctgcctatcctgacggaaggaaaggaaaaagcgccacgtccagctcccacaccgcaggctccagcgcaaaatacaacgccggtcacaaaaacgcgtttaatagataccccggtgcgttccggtcagcgtatttatgctccacaatgtgatctgattgttacaagccacgttagcgctggggccgaattgattgccgatgggaacattcatgtctatggcatgatgcgcggtcgtgcgctggcaggggcaagtggtgaccgggaaacgcaaatattttgtacgaacctgatggcggaactggtgtccatcgcaggtgaatactggctgagtgatcaaatcccagcagaattttatggcaaagcggcgcgactgcagttagtcgaaaacgctttgaccgttcaaccgttaaattga

SEQ ID NO: 2 - protein, 231 aa, Escherichia coli
MSNTPIELKGSSFTLSVVHLHEAEPKVIHQALEDKIAQAPAFLKHAPVVLNVSALEDPVNWSAMHKAVSATGLRVIGVSGCKDAQLKAEIEKMGLPILTEGKEKAPRPAPTPQAPAQNTTPVTKTRLIDTPVRSGQRIYAPQCDLIVTSHVSAGAELIADGNIHVYGMMRGRALAGASGDRETQIFCTNLMAELVSIAGEYWLSDQIPAEFYGKAARLQLVENALTVQPLN

SEQ ID NO: 3 - DNA, 813 bp, Escherichia coli
atggcacgcattattgttgttacttcgggcaaagggggtgttggtaagacaacctccagcgcggccatcgccactggtttggcccagaagggaaagaaaactgtcgtgatagattttgatatcggcctgcgtaatctcgacctgattatgggttgtgaacgccgggtcgtttacgatttcgtcaacgtcattcagggcgatgcaacgctaaatcaggcgttaattaaagataagcgtactgaaaatctctatattctgccggcatcgcaaacacgcgataaagatgccctcacccgtgaaggggtcgccaaagttcttgatgatctgaaagcgatggattttgaatttatcgtttgtgactccccggcagggattgaaaccggtgcgttaatggcactctattttgcagacgaagccattattaccaccaacccggaagtctcctcagtacgcgactctgaccgtattttaggcattctggcgtcgaaatcacgccgcgcagaaaatggcgaagagcctattaaagagcacctgctgttaacgcgctataacccaggccgcgtaagcagaggtgacatgctgagcatggaagatgtgctggagatcctgcgcatcaaactcgtcggcgtgatcccagaggatcaatcagtattgcgcgcctctaaccagggtgaaccggtcattctcgacattaacgccgatgcgggtaaagcctacgcagataccgtagaacgtctgttgggagaagaacgtcctttccgcttcattgaagaagagaagaaaggcttcctcaaacgcttgttcggaggataa

SEQ ID NO: 4 - protein, 270 aa, Escherichia coli
MARIIVVTSGKGGVGKTTSSAAIATGLAQKGKKTVVIDFDIGLRNLDLIMGCERRVVYDFVNVIQGDATLNQALIKDKRTENLYILPASQTRDKDALTREGVAKVLDDLKAMDFEFIVCDSPAGIETGALMALYFADEAIITTNPEVSSVRDSDRILGILASKSRRAENGEEPIKEHLLLTRYNPGRVSRGDMLSMEDVLEILRIKLVGVIPEDQSVLRASNQGEPVILDINADAGKAYADTVERLLGEERPFRFIEEEKKGFLKRLFGG

SEQ ID NO: 5 - DNA, 267 bp, Escherichia coli
atggcattactcgatttctttctctcgcggaagaaaaacacagccaacattgcaaaagaacggctgcagattattgttgctgaacgccgtcgcagcgatgcagaaccgcattatctgccgcagttgcgtaaagatattcttgaggtcatttgtaaatacgtacaaattgatcctgagatggtaaccgtacagcttgagcaaaaagatggcgatatttctattcttgagctgaacgtgaccttaccggaagcagaagagctgaaataa

SEQ ID NO: 6 - protein, 88 aa, Escherichia coli
MALLDFFLSRKKNTANIAKERLQIIVAERRRSDAEPHYLPQLRKDILEVICKYVQIDPEMVTVQLEQKDGDISILELNVTLPEAEELK

SEQ ID NO: 7 - DNA, 120 bp, Artificial Sequence
  Artificially synthesized oligonucleotide
gtgacttgcctcaatataatccagactataacatgccttatagtcttcggaacatcatcgcgcgctggcgatgattaatagctaattgagtaaggccagggtgtaggctggagctgcttc

SEQ ID NO: 8 - DNA, 120 bp, Artificial Sequence
  Artificially synthesized oligonucleotide
cgctgcgacggcgttcagcaacaataatctgcagccgttcttttgcaatgttggctgtgtttttcttccgcgagagaaagaaatcgagtaatgccataacatgggaattagccatggtcc

SEQ ID NO: 9 - DNA, 24 bp, Artificial Sequence
  Artificially synthesized oligonucleotide
gattgaacaagatggattgcacgc

SEQ ID NO: 10 - DNA, 24 bp, Artificial Sequence
  Artificially synthesized oligonucleotide
ctcgtcaagaaggcgatagaaggc

SEQ ID NO: 11 - DNA, 3393 bp, Artificial Sequence
  Artifically constructed plasmid pRAIDA2
ggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaagtcatctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgcgcagaaaggcccacccgaaggtgagccaggtgattacatttgggccctcattagaaaaactcatcgagcatcaaatgaaattgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatagctcctgaaaatctcgataactcaaaaaatacgcccggtagtgatcttatttcattatggtgaaagttggaacctcttacgtgccgatcaaaaagacggtcaaaagcctccggtcggaggcttttgactttctgctatggaggtcaggtatgatttaaatggtcagtattgagcgatatctagagaattcgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaggagatatacataggtctcacatgattaaattaaaatttggtgttttttttacagttttactatcttcagcatatgcacatggaacacctcaaaatattactcacggtgtgttaaccctgtcttctcgtggtctggactatccatacgatgtaccggattacgcgcgttatggaagacgagtgttaacaatggagatcttcgaacaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaagcttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttctgcaggtgggggatataacctgaatgtgcacacatggacatcacctgaaggaataacaggtgaattctggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggatccataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttcggttagagc

(19) United States
(12) Patent Application Publication
(10) Pub. No.: US 2024/0309390 A1
(43) Pub. Date: Sep. 19, 2024

(54) MINICELLS FROM HIGHLY GENOME REDUCED ESCHERICHIA COLI: CYTOPLASMIC AND SURFACE EXPRESSION OF RECOMBINANT PROTEINS AND INCORPORATION IN THE MINICELLS

(71) Applicant: University of Virginia Patent Foundation, Charlottesville, VA (US)

(72) Inventors: Steven L. Zeichner, Bethesda, MD (US); Mark Kester, Afton, VA (US)

(73) Assignee: University of Virginia Patent Foundation, Charlottesville, VA (US)

(21) Appl. No.: 18/566,497

(22) PCT Filed: Jun. 1, 2022
(86) PCT No.: PCT/US2022/031807

§ 371 Date: Dec. 1, 2023

Related U.S. Application Data
(60) Provisional application No. 63/195,482, filed on Jun. 1, 2021.

(51) Int. Cl. C12N 15/70; A61K 39/00; C12N 1/20

(52) U.S. Cl. CPC C12N 15/70; C12N 1/20; A61K 39/00

(57) ABSTRACT

Provided are bacterial minicells derived from genome reduced (GR) having a reduced number of expressed genes and/or is a bacterium having one or more mutated min genes. In some embodiments, the minicell has a recombinant protein present in and/or on the surface of the minicell. In some embodiments, the recombinant protein is an antigen and in some embodiments, the minicell induces an enhanced immune response against the antigen when administered to a subject. In some embodiments, the bacterium has an autotransporter (AT) expression vector encoding the recombinant protein to express the recombinant protein on the surface of the bacterium and/or the minicell derived therefrom. Also provided are vaccine compositions that include bacterial minicells, methods for producing antibodies, methods for vaccinating subjects, and expression vectors encoding heterologous proteins.

### CROSS REFERENCE TO RELATED APPLICATION

The presently disclosed subject matter claims the benefit of U.S. Provisional Patent Application Ser. No. 63/195,482, filed Jun. 1, 2021, the disclosure of which incorporated herein by reference in its entirety.

### REFERENCE TO SEQUENCE LISTING SUBMITTED ELECTRONICALLY

The content of the electronically submitted sequence listing in ASCII text file (Name: 1586_24_PCT_ST25.txt; Size: 14 kilobytes; and Date of Creation: Jun. 1, 2022) filed with the instant application is incorporated herein by reference in its entirety.

### TECHNICAL FIELD

The presently disclosed subject matter relates to compositions and methods useful in the context of drug delivery, vaccine development, and antibody production. In representative embodiments, the presently disclosed subject matter relates to minicells, small cells lacking a chromosome, produced by bacteria with mutated min genes and bacteria modified to have reduced expression of genes, such as by having a reduction of the bacterial genomes and, in some embodiments, using those bacteria to express antigens of interest.

### BACKGROUND

The development of new, faster, more effective methods to prepare recombinant proteins, such as might be used to induce immune responses in subjects represents a need in the art. The presently disclosed subject matter addresses these and other needs in the art.

Effective vaccines exist to prevent and treat some human and animal diseases, but there are no vaccines for other important infections of humans and animals. Some existing vaccines do not provide for the rapid development of an effective immune response, and new infectious agents, both naturally occurring infectious agents and maliciously disseminate agents, continue to emerge. Current approaches to inducing immune responses against cancers are slow, cumbersome, and expensive. Many pathologic processes, such as autoimmune and inflammatory diseases result from the dysregulation or inappropriate expression of components of the immune system or inflammatory mediators. Inflammatory and autoimmune diseases can be treated with agents that target inflammatory mediators and components of the immune system. A method to develop more effective vaccines, vaccines that more rapidly induce an immune response against pathogens, cancers, and components of the immune system and mediators of inflammatory processes, and vaccines that be produced quickly in response to new biological threats is urgently needed.

The presently disclosed subject matter addresses these needs in the art and society.

### SUMMARY

This summary lists several embodiments of the presently disclosed subject matter, and in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to bacterial minicells derived from a bacterium that is a genome reduced (GR) bacteria having a reduced number of expressed genes and/or is a bacterium that has one or more mutated min genes. In some embodiments, and/or is a GR bacterium having one or more mutated min genes. In some embodiments, the one or more mutated min genes is/are a minC gene and/or a minD gene.

In some embodiments, the minicell comprises a recombinant protein. In some embodiments, the recombinant protein is present in cytoplasm and/or on a surface of the minicell. In some embodiments, the recombinant protein is present in the minicell in an enhanced amount as compared to an amount that would have been present in a minicell derived from a bacterium of the same strain that has a full complement of expressed genes and/or an unmutated min gene.

In some embodiments, the recombinant protein is an antigen, optionally an antigen on a surface of a membrane, or a derivative thereof. In some embodiments, the minicell induces an enhanced immune response against the antigen when administered to a subject as compared to an immune response that would have been induced in the subject by a minicell derived from a bacterium of the same strain that has a full complement of expressed genes and/or an unmutated min gene.

In some embodiments, reducing and/or eliminating expression of one or more genes and/or mutating one or more min genes in the bacterium yields the enhanced immune response.

In some embodiments, the bacterium is a Gram-negative bacterium, optionally a member of the Enterobacteriaceae. In some embodiments, the bacterium is an E. coli bacterium, a Shigella bacterium, a Yersinia bacterium, or a Salmonella bacterium. In some embodiments, the bacterium is an E. coli bacterium.

In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some embodiments, the recombinant protein, optionally the antigen, is put on the surface of the minicell by an approach selected from the group consisting of expression by the bacterium, covalent or non-covalent association with the outer membrane, and combinations thereof.

In some embodiments, the bacterium comprises an autotransporter (AT) expression vector encoding the recombinant protein, optionally the antigen, wherein the expression on the surface is provided by the AT expression vector. In some embodiments, the AT expression vector comprises a codon optimized sequence encoding the antigen. In some embodiments, the AT expression vector comprises a monomeric autotransporter vector or a trimeric autotransporter vector.

In some embodiments, the recombinant protein, optionally the antigen, is derived from a microbe. In some embodiments, the recombinant protein, optionally the antigen, is derived from a tumor and/or a cancer, a target of an inappropriate or undesirable immune response, or a component of the host immune system, optionally a host immune system component that, when targeted for destruction, inactivation, or activation, alters an undesirable immune response.

In some embodiments, the presently disclosed also relates to methods for producing an antibody in a subject. In some embodiments, the methods comprise, consist essentially of, or consist of providing a minicell of the presently disclosed subject matter and administering the minicell to a subject in an amount and via a route sufficient to produce an antibody in the subject against the antigen present in and/or on the minicell. In some embodiments, the production of the antibody is enhanced in the subject as compared to that which would have been induced in the subject by a minicell derived from a bacterium of the same strain that has a full complement of expressed genes and/or an unmutated min gene. In some embodiments, the minicell is administered to the subject intranasally, transmucosally, including but not limited to orally, rectally, and vaginally; subcutaneously, intradermally, intramuscularly, other parenteral routes, or any combination thereof.

In some embodiments, the presently disclosed subject matter also relates to vaccine compositions comprising, consisting essentially of, or consisting of one or more bacterial minicells as described herein and a pharmaceutically acceptable carrier. In some embodiments, the vaccine composition further comprises, consists essentially of, or consists of one or more adjuvants. In some embodiments, the vaccine composition is adapted to be administered orally, rectally, vaginally, intra-nasally, parenterally, intradermally, subcutaneously, or intramuscularly.

In some embodiments, the presently disclosed subject matter also relates to methods for vaccinating subjects in need thereof. In some embodiments, the methods comprise, consist essentially of, or consist of providing a vaccine composition of the presently disclosed subject matter and administering the vaccine composition to the subject.

In some embodiments, the presently disclosed subject matter also relates to methods for treating a tumor and/or a cancer and/or an inappropriate immune response and/or expression and/or production of a deleterious material in subjects in need thereof. In some embodiments, the methods comprise, consist essentially of, or consist of providing a vaccine composition of the presently disclosed subject matter and administering the vaccine to the subject.

In some embodiments, a vaccine composition of the presently disclosed subject matter is administered orally, rectally, vaginally, intra-nasally, parenterally, intradermally, subcutaneously, or intramuscularly.

In some embodiments, the presently disclosed subject matter also relates to expression vectors comprising a nucleotide sequence encoding a heterologous protein. Ise, the expression vector is configured to express the heterologous protein in and/or on the surface of a minicell derived from a modified bacterium having a reduced number of expressed genes and/or one or more mutated min genes. In some embodiments, the heterologous protein is an antigen. In some embodiments, the vector comprises a codon optimized sequence encoding the antigen. In some embodiments, the expression vector is an autotransporter (AT) expression vector. In some embodiments, the AT expression vector comprises a monomeric vector or a trimeric vector. In some embodiments, the nucleotide sequence encoding the heterologous protein, optionally an antigen, is positioned under control of an inducible promoter or a constitutive promoter. In some embodiments, the heterologous protein, optionally an antigen, is expressed as a monomer or as a trimer. In some embodiments, the expression vector is provided in a pharmaceutically acceptable carrier.

Accordingly, it is an object of the presently disclosed subject matter to provide minicells, methods employing minicells, and compositions comprising minicells.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTIONS OF THE FIGURES

FIG. 1. pRAIDA2 plasmid map. The pRAIDA2 plasmid has a high copy origin of replication (ori), a kanamycin resistance gene (KanR), an AIDA-I autotransporter under the control of a rhamnose inducible promoter (PrhaBAD), and a transcriptional terminator (Term). The parental version of pRAIDA2 enables the expression of an influenza HA immunotag (HA-tag) on the surface of a bacterial cell. The sequence of pRAIDA2 has been deposited into the GENBANK® biosequence database (referred to therein as “Synthetic construct pRAIDA2”) and has been assigned Accession No. MW383928.1 (SEQ ID NO: 11).

FIGS. 2A-2C. Immunoblots for recombinant proteins expressed in the cytoplasm (GFP) and on the surface (autotransporter-expressed HA immunotag) of non-highly genome reduced and highly genome-reduced bacteria, and minicells produced by the bacteria. FIG. 2A. Bacteria expressing GFP in the cytoplasm. Indicated lanes show the DNAK control normalization protein and the recombinant cytoplasmically expressed GFP in bacteria and minicells produced from the bacteria for the undeleted ME 5000 minC minD strain and the 29.7% deleted ME 5125 minC minD strain. FIG. 2B. Bacteria expressing HA via the AIDA-I autotransporter surface expression cassette. Indicated lanes show the DNAK control normalization protein and the HA immunotag in bacteria and minicells produced from the bacteria for the undeleted ME 5000 minC minD strain and the 29.7% deleted ME 5125 minC minD strain. FIG. 2C. Fold enrichment for the signal due to the indicated proteins, normalized to DNAK, in the minicell preparation compared to the bacteria.

FIGS. 3A-3D. Examples of images and analysis of image cytometry data from ME 5125 minC minD expressing either the cytoplasmic protein GFP or HA expressed via the AIDA-I autotransporter, showing an apolar or polar appearance. FIG. 3A. Apolar cells expressing GFP. FIG. 3B. Apolar cells expressing HA. FIG. 3C. Polar cells expressing GFP. FIG. 3D. Polar cells expressing HA via the AIDA-I autotransporter expression cassette. Of note, similarly to data described in Jain et al., 2006, for experiments involving the AIDA-I-expressed HA immunotag, the signal due to the autotransporter-expressed HA immunotag, concentrates at the poles of the bacteria (FIG. 3B), and then in these minC minD minicell producing mutant bacteria, appears to be further concentrated in the budding structures at the bacterial poles that are the precursors to minicells (FIG. 3D).

FIGS. 4A-4H. Image cytometry gating and analysis of differences in expression of the cytoplasmic protein, GFP, or the HA immunotag expressed via the AIDA-I autotransporter surface expression cassette. FIG. 4A. Image cytometry analysis of ME 5125 without an expressing plasmid. FIG. 4B. Image cytometry analysis of ME 5125 expressing GFP. FIG. 4C. Image cytometry analysis of ME 5125 expressing HA on the bacterial surface. FIG. 4D. Image cytometry analysis of ME 5125 minC minD without an expressing plasmid. FIG. 4E. Image flow analysis of ME 5125 minC minD expressing GFP. FIG. 4F. Image cytometry analysis of ME 5125 minC minD expressing HA on the bacterial surface. FIG. 4G. The ratio of the signal intensity from the fluorescence due to the recombinant protein GFP to the area of the gated particle (“Hi scatter” for the elongated bacteria, “Lo scatter” for the small, close-to-spherical minicells) in the preparation (arbitrary units), which contains both bacteria and derived minicells, for either the undeleted ME 5000 minC minD bacteria or the 29.7% deleted ME 5125 minC minD bacteria, as indicated. FIG. 4H. The ratio of the signal intensity from the fluorescence due to the recombinant HA protein to the area of the gated particle (“Hi scatter” for the elongated bacteria, “Lo scatter” for the small, close-to-spherical minicells) in the preparation (arbitrary units), which contains both bacteria and derived minicells, for either the undeleted ME 5000 minC minD bacteria or the 29.7% deleted ME 5125 minC minD bacteria, as indicated.

FIGS. 5A and 5B. Examples of image cytometry images of minicells isolated from undeleted ME 5000 minC minD or 29.7% deleted ME 5125 minC minD. FIGS. 5A and 5B show examples of two minicells analyzed by image cytometry from undeleted ME 5000 minC minD (FIG. 5A) or 29.7% deleted ME 5125 minC mind (FIG. 5B), expressing either GFP or HA, as indicated. Each triple image shows a brightfield captured image (top), a fluorescent image of the minicell's recombinant protein (middle), and a merged image (bottom).

FIGS. 6A-6D. Minicell size determination. Isolated minicells were made from E. coli ME 5125 minC minD expressing either GFP or HA via the AIDA-I autotransporter in pRAIDA2. FIG. 6A. Size determination for minicells made from E. coli ME 5125 minC minD expressing GFP, as determined by quantitation of Amnis ImageStream images. FIG. 6B. Size determination for minicells made from E. coli ME 5125 minC minD expressing GFP, as determined by DLS. FIG. 6C. Size determination for minicells made from E. coli ME 5125 minC minD expressing HA, as determined by quantitation of Amnis ImageStream images.

FIG. 6D, Size determination for minicells made from E. coli ME 5125 minC minD expressing HA, as determined by DLS. Note that the size measurement outputs from the DLS instrument and Amnis ImageStream instrument are scaled differently (log vs. linear), but the peaks appear at close to the same values, approximately 800 nM.

### DETAILED DESCRIPTION

### I. General Considerations

Investigators have studied the minimum complement of genes required for living bacteria. Some investigators have taken an additive, synthetic approach, to produce a bacterial cell containing a carefully curated complement of genes (Zhang et al., 2010; Juhas, 2016; Sung et al., 2016). Other investigators have taken a subtractive approach, in which they make serial deletions in the bacterial chromosome and then show that the bacteria with the deleted chromosome still live. The Tokyo Metropolitan University Group, for example (Hashimoto et al., 2005; Kato & Hashimoto, 2007), showed that they could delete up to 29.7% of the E. coli genome and still have viable bacteria. Other investigators have deleted about 40% of the Bacillus subtilis genome (Reuß et al., 2017). GR bacteria can exhibit altered growth characteristics, however. For E. coli, the doubling time is about twice as long for the highly deleted strains than for the wild type, and the highly deleted strains exhibit altered morphology. Such studies offer useful insights into the minimal complement of genes needed to direct a viable bacterial cell.

While the production of genome reduced (GR) bacteria is interesting because it addresses basic biological questions, the GR bacteria also present useful biotechnological applications. GR bacteria also present useful biotechnological applications, for example, by facilitating the generation of “difficult to produce proteins” (Aguilar Suárez et al., 2019) or enhancing the production of antimicrobial peptides like lantibiotics (van Tilburg et al., 2020). Eliminating genes not essential for growth can minimize diversion of energy and substrates for biotechnologically nonproductive purposes or make additional chromosome capacity available for engineering goals. Eliminating genes involved in formation of bacterial structures may enable more effective utility of remaining structures. If animals are exposed to antigen overexpressing bacteria with many fewer functional genes, they have the potential to be less reactogenic.

Minicells (reviewed in Farley et al., 2016) were described more than 50 years ago (Adler et al., 1967). In E. coli, the proteins of the Min system—MinC, MinD, and MinE—control the placement of the Z-ring in the middle of the bacterial cell by preventing assembly of the FtsZ complex at locations other than the middle of the cell. In wild type cells, these proteins promote division of the cell into approximately equal size daughter cells, helping to prevent formation of daughter cells lacking a bacterial chromosome. In min mutant cells, the Z-ring can form not only in the middle of the cell about to undergo division, but also toward one of the poles of the cell. When the Z-ring forms close to a bacterial pole, cell division yields a large cell with copies of the bacterial chromosome and a minicell lacking the bacterial chromosome. Mother cells containing bacterial chromosomes continue to divide, enabling ongoing production of minicells. While the daughter minicells are incapable of further reproduction, they remain metabolically and biosynthetically active. Minicells can be readily produced in quantity, using differential centrifugation and filtration approaches (Shepherd et al., 2001; MacDiarmid et al., 2007).

Minicells have proved useful, both for the study of basic biological processes and for biotechnological uses. Studies employing cryoelectron tomography are more easily conducted with smaller particles, so minicells have been helpful in the study of bacterial processes and machinery, such as the flagellum and Type III secretion systems (Macnab, 2003; Schraidt & Marlovits, 2011). Minicells have also played an important part in studies of bacteriophage physiology (Hu et al., 2013; Sun et al., 2014; Hu et al., 2015). Biotechnological applications include the use of minicells to encapsulate drugs (MacDiarmid et al., 2007), and have been proposed as potential vaccine antigen delivery vehicles (Giacalone et al., 2006; Charlotte, 2013).

Recombinant proteins can be expressed in the bacterial cytoplasm with relative ease. However, it may also be useful to place recombinant proteins on bacterial surfaces and make bacterial derivatives with enriched concentrations of recombinant proteins. Gram-negative autotransporters (autodisplay proteins, Type V secretion systems; see e.g., Jose & Meyer, 2007; van Ulsen et al., 2018; Meuskens et al., 2019) enable the placement of large numbers of recombinant proteins on the bacterial surface. Autotransporters have three domains: an N-terminal signal sequence that helps mediate transfer of the protein across the inner membrane via a Sec translocon mechanism, a central passenger protein domain that includes the effector portion of the protein, and a C-terminal β-barrel domain that intercalates into the outer membrane to form a pore-like structure, aided by the β-barrel assembly machinery (Bam) complex (Ieva & Bernstein, 2009). The passenger domain transits out to the extracellular environment through the pore of the β-barrel. The β-barrel may exhibit chaperonin-like activity, aiding in the correct formation of passenger protein tertiary structure during transit to the extracellular environment. Subject to some limitations, for example an intolerance for disulfide bonds and certain size limitations, DNA sequences encoding heterologous proteins can replace native passenger protein coding sequence so that autotransporters can be used to place heterologous recombinant proteins on the surface of the bacteria exposed to the extracellular environment, anchored into the outer membrane by the β-barrel. Recombinant autotransporters have been used to place a variety of biotechnologically useful molecules on the surfaces of bacteria, including enzymes, biosensors, and vaccine antigens. However, no clinically useful vaccines have yet been produced using autotransporter expression systems, perhaps because the first vaccine applications were attempted using live bacteria as vaccine vectors with attendant safety concerns (Jose & Meyer, 2007; van Ulsen et al., 2018), or the antigens expressed on the surfaces of the bacteria were insufficiently immunogenic. Surface expression of antigens on minicells derived from GR bacteria may address these limitations.

At least some autotransporters preferentially accumulate at the bacterial poles (Jain et al., 2006), so it may be anticipated that recombinant proteins expressed on the surfaces of bacteria via autotransporters will be enriched in minicells, since the minicells bud off from the bacterial poles. Combining substantial genome reduction with expression of recombinant proteins on the bacterial surface using autotransporters in combination with budding of the minicells from the bacterial poles would be expected to additionally enrich the relative amounts of the recombinant proteins on the surface of the minicells compared to expression of a protein in the cytoplasm or on the surface of non-genome reduced whole bacterial cells. Although work has been done with outer membrane vesicles that include heterologous proteins placed on their surfaces using autotransporters (Daleke-Schermerhorn et al., 2014; Jong et al., 2014; Kuipers et al., 2015), we are not aware of work that describes the use of minicells made from bacteria expressing recombinant proteins on their surfaces using autotransporter-mediated expression. While both genome-reduced bacteria and minicells hold significant promise for a variety of biotechnological purposes, to our knowledge, these two technologies have not been previously combined. Here, we show that the minC and minD genes of highly GR E. coli can be deleted and that the resulting mutant bacteria are viable and biotechnologically functional. The minC/minD GR E. coli produce minicells with typical minicell characteristics, and recombinant proteins can be expressed by the minC/minD GR E. coli in the cytoplasm and on the surface of minicells made from the GR E. coli. An AIDA-I-mediated surface expressed recombinant protein was enriched on the minicells compared to the parental non-minicell-producing E. coli. Expressing recombinant protein in the cytoplasm or on the surface of minicells made from genome reduced bacteria can provide several new, useful biotechnological and biomedical applications including vaccines with enhanced immunogenicity and less toxic vehicles for targeted drug delivery.

### II. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 20%, 15%, 10%, 5%, or 1%.

A disease, disorder, and/or condition is “alleviated” if the severity of a symptom of the disease, disorder, and/or condition or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, disorder, and/or condition being treated. Such a compound, for example, could include one being used to treat an unrelated disease, disorder, and/or condition, and/or a disease, disorder, and/or condition\which may not be responsive to the primary treatment for the injury, disease, disorder, and/or condition being treated.

As used herein, the term “adjuvant” refers to a substance that elicits an increased immune response when used in combination with a specific antigen.

As use herein, the terms “administration of” and or “administering” a compound should be understood to mean providing a compound of the presently disclosed subject matter and/or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in the following Table 1:

TABLE 1
Summary of Amino Acids and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The term “amino acid” is used interchangeably with “amino acid residue”, and may refer to a free amino acid and to an amino acid residue of a peptide. It will be apparent from the context in which the term is used whether it refers to a free amino acid or a residue of a peptide. Amino acids have the following general structure:

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:
I. Small aliphatic, nonpolar, or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys
V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab, and F(ab)₂ fragments, as well as single chain antibodies and humanized antibodies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates. The term “immunogen” is used interchangeably with “antigen” herein.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat and urine.

As used herein, the term “carrier molecule” refers to any molecule that is chemically conjugated to the antigen of interest that enables an immune response resulting in antibodies specific to the native antigen.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes, or contributes to a disease or disorder in the animal in which the tissue is located (or from which the tissue was obtained).

A tissue “normally comprises” a cell if one or more of the cell are present in the tissue in an animal not afflicted with a disease or disorder.

As used herein, a “derivative” of a bacterium, antigen, composition, or other compound refers to a bacterium, antigen, composition, or other compound that may be produced from bacterium, antigen, composition, or other compound of similar structure in one or more steps.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly at least five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99% by weight of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be in some embodiments at least about 20 nucleotides in length, in some embodiments at least about 50 nucleotides, in some embodiments from about 50 to about 100 nucleotides, in some embodiments at least about 100 to about 200 nucleotides, in some embodiments at least about 200 nucleotides to about 300 nucleotides, in some embodiments at least about 300 to about 350 nucleotides, in some embodiments at least about 350 nucleotides to about 500 nucleotides, in some embodiments at least about 500 to about 600 nucleotides, in some embodiments at least about 600 nucleotides to about 620 nucleotides, in some embodiments at least about 620 to about 650 nucleotides, and in some embodiments the nucleic acid fragment will be greater than about 650 nucleotides in length.

The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container which contains the identified compound presently disclosed subject matter or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

As used herein, the term “minC” refers to the septum site-determining protein MinC genetic locus and gene products derived therefrom, exemplified by the Escherichia coli minC. An exemplary amino acid sequence of an Escherichia coli minC gene product is described, for example, in Accession No. NP_415694.1 of the GENBANK® biosequence database (SEQ ID NO: 2). This Escherichia coli minC polypeptide is encoded by the reverse complement of nucleotides 1,225,385-1,226,080 of Accession No. NC_000913.3 of the GENBANK® biosequence database and is provided as SEQ ID NO: 1. Non-limiting examples of minC orthologs from other species include those set forth in Accession Nos. RXX49312.1 (Klebsiella pneumoniae), MCB2606493.1 (Listeria monocytogenes), MXH34759.1 (Pseudomonas aeruginosa), WP_119170622.1 (Shigella dysenteriae), ACD10196.1 (Shigella boydii), EGA1647163.1 (Shigella sonnei), WP_039060698.1 (Shigella flexneri), WP_059228803.1 (Escherichia albertii), WP_187221688.1 (Escherichia marmotae), MBY7308686.1 (Escherichia ruysiae), WP_121584735.1 (Citrobacter), EGT0649697.1 (Citrobacter braakii), HAT2257996.1 (Citrobacter freundii), WP_239605304.1 (Citrobacter portucalensis), MBJ3557328.1 (Salmonella enterica subsp. enterica serovar Derby), and WP_002220631.1 (Yersinia) of the GENBANK® biosequence database.

As used herein, the term “minD” refers to the septum site-determining protein MinD genetic locus and gene products derived therefrom, exemplified by the Escherichia coli minD. An exemplary amino acid sequence of an Escherichia coli minD gene product is described, for example, in Accession No. NP_415693.1 of the GENBANK® biosequence database (SEQ ID NO: 4). This Escherichia coli minD polypeptide is encoded by the reverse complement of nucleotides 1,224,549-1,225,361 of Accession No. NC_000913.3 of the GENBANK® biosequence database and is provided as SEQ ID NO: 3. Non-limiting examples of minD orthologs from other species include those set forth in Accession Nos. WP_124783238.1 (Escherichia albertii), WP_000101046.1 (Escherichia fergusonii), WP_042287710.1 (Citrobacter), WP_103774789.1 (Citrobacter amalonaticus), WP_249574986.1 (Citrobacter cronae), EJA2599346.1 (Citrobacter farmeri), WP_125368248.1 (Citrobacter sedlakii), WP_070262566.1 (Listeria monocytogenes), MXH34758.1 (Pseudomonas aeruginosa), WP 000101045.1 (Salmonella bongori), WP_024797690.1 (Salmonella enterica), EDV1004335.1 (Salmonella enterica subsp. enterica), HAT5387785.1 (Salmonella enterica subsp. enterica serovar Typhimurium), WP_073809863.1 (Shigella boydii), EFY0240827.1 (Shigella dysenteriae), EFW2239911.1 (Shigella flexneri), EFV8337658.1 (Shigella sonnei), and WP_002211179.1 (Yersinia) of the GENBANK® biosequence database.

As used herein, the term “minE” refers to the septum site-determining protein MinE genetic locus and gene products derived therefrom, exemplified by the Escherichia coli minE. An exemplary amino acid sequence of an Escherichia coli minE gene product is described, for example, in Accession No. Accession No. NP_415692.1 of the GENBANK® biosequence database (SEQ ID NO: 6). This Escherichia coli minE polypeptide is encoded by the reverse complement of nucleotides 1,224,279-1,224,545 of Accession No. NC_000913.3 of the GENBANK® biosequence database and is provided as SEQ ID NO: 5. Exemplary minE orthologs from other species include those set forth in Accession Nos. WP_096756724.1 (Citrobacter), HBB6752258.1 (Citrobacter freundii), WP_105197759.1 (Escherichia albertii), WP_155108765.1 (Intestinirhabdus alba), WP_138160244.1 (Klebsiella), WP_064544824.1 (Kluyvera), WP_103180012.1 (Leclercia), WP_042391279.1 (Pseudescherichia vulneris), MXH34757.1 (Pseudomonas aeruginosa), WP_219123888.1 (Salmonella enterica), ECI3802950.1 (Salmonella enterica subsp. enterica), EDL8520673.1 (Salmonella enterica subsp. enterica serovar Derby), KJT57442.1 (Salmonella enterica subsp. enterica serovar Heidelberg), WP_039060697.1 (Shigella flexneri), HAW4533521.1 (Shigella sonnei), PTF44438.1 (Staphylococcus epidermidis), WP_038153586.1 (Trabulsiella), and WP_002211180.1 (Yersinia) of the GENBANK® biosequence database.

The term “nasal administration” in all its grammatical forms refers to administration of at least one composition of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, trans-mucosal administration of a drug is highly amenable to self administration, and intranasal administration of antigens exposes the antigen to a mucosal compartment rich in surrounding lymphoid tissues, which can promote the development of a more potent immune response, particularly more potent mucosal immune responses.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments a human.

As used herein, a “subject in need thereof” is a patient, animal, mammal, or human, who will benefit from the method of this presently disclosed subject matter.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Exemplary, non-limiting nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; 7% (SDS), 0.5 M NaPO₄, 1 mM EDTA at 50° C. with washing in 1×SSC, 0.1% SDS at 50° C.; 7% SDS, 0.5 M NaPO₄, 1 mM EDTA at 50° C. with washing in 0.5×SSC, 0.1% SDS at 50° C.; and 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990b; Altschul et al., 1990a; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “treat” and grammatical variants thereof, as used herein, means reducing the frequency and/or severity with which symptoms are experienced by a patient or subject or administering an agent or compound to reduce the frequency and/or severity with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine”, as used herein, is meant a composition which when inoculated into a subject has the effect of stimulating an immune response in the subject, which serves to fully or partially treat and/or protect the subject against a disease, disorder, and/or condition, or a symptom thereof. In some embodiments, the disease, disorder, and/or condition is caused by a microbe (e.g., a viral disease including but not limited to HIV, COVID-19, MERS, etc.). TB is another application as are parasitic diseases. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### III. Representative Embodiments

Minicells, small cells lacking a chromosome, in some embodiments produced by bacteria with mutated min genes, which control cell division septum placement, have many potential uses. Minicells have contributed to basic bacterial physiology studies and can enable new biotechnological applications, including drug delivery and vaccines.

Genome-reduced (GR) bacteria are another informative area of investigation. Investigators identified that with even almost 30% of the E. coli genome deleted, the bacteria still live. In biotechnology and synthetic biology, GR bacteria offer certain advantages. With GR bacteria, more recombinant genes can be placed into GR chromosomes and fewer cell resources are devoted to purposes apart from biotechnological goals. Here, it is shown that these two technologies can be combined: min mutants can be made in GR E. coli. The minC minD mutant GR E. coli produce minicells that concentrate engineered recombinant proteins within these spherical delivery systems. Recombinant GFP protein was expressed in the cytoplasm of GR bacteria and showed that it was concentrated within the minicells. Proteins were also expressed on the surfaces of minicells made from GR bacteria using a recombinant Gram-negative AIDA-I autotransporter expression cassette. Some autotransporters, like AIDA-I, are concentrated at the bacterial poles where minicells bud, and because the surface-to-volume ratio of the small minicells is higher than that of the corresponding non-GR bacteria, recombinant proteins expressed on surfaces of the GR bacteria are concentrated on the minicells. Minicells made from GR bacteria thus facilitate useful biotechnological innovations, such as by acting as drug delivery vehicles and delivering vaccine immunogens.

While the presently disclosed subject matter exemplifies minicells produced from E. coli, minicells can also be produced from other bacteria, such as but not limited to Shigella, Pseudomonas, Yersinia, Corynebacterium, and Salmonella. See Farley et al., 2016. By way of example and not limitation, inactivation of Min or Min-like systems has proven successful in generating minicells from Salmonella enterica (Carleton et al., 2013), Pseudomonas aeruginosa (MacDiarmid et al., 2007), and Corynebacterium glutamicum (Lee et al., 2015).

However, in some bacterial species, inactivation of the Min system results in lower minicell yields. For example, inactivation of the Listeria monocytogenes Min system produces minicells at very low frequency (Kaval et al., 2014), perhaps because of the action of additional Z-ring spatial regulators in this species.

Alternatively or in addition, the overproduction of FtsZ can overwhelm the Min system and produce minicells in an otherwise wild-type strain of bacteria, including but not limited to E. coli (Ward Jr. & Lutkenhaus, 1985). A similar strategy was recently used to overproduce FtsZ and resulted in a 2-fold increase in minicell production of L. monocytogenes (Kaval et al., 2014). Thus, in some embodiments minicells are produced by overexpressing FtsZ gene products in bacteria. Exemplary FtsZ polypeptide gene products are disclosed in the GENBANK® biosequence database, and include the following Accession Nos.: NP 414637.1 (Escherichia coli), NP_389412.2 (Bacillus subtilis subsp. subtilis), NP_459138.1 (Salmonella enterica subsp. enterica serovar Typhimurium), NP_253097.1 (Pseudomonas aeruginosa), NP_465556.1 (Listeria monocytogenes), and WP_003856520.1 (Corynebacterium glutamicum).

As is known in the art, in some embodiments multiple gene products can be generated from a particular genetic locus, for example by alternative transcriptional initiation sites, alternative splicing, etc. It is understood that the GENBANK® Accession Nos. presented herein are meant to be exemplary only, and other gene products for which the nucleotide and/or amino acid sequences are not explicitly disclosed herein are also intended to be encompassed by the names of the corresponding genes. Thus, for example, transcript variants of the sequences in the Sequence Listing are also included with the definitions of the genes described herein, as are the amino acid variants encoded thereby.

In some embodiments, the presently disclosed subject matter provides a bacterial minicell derived from a bacterium having a reduced number of expressed genes as compared to a wild type bacteria and/or one or more mutated min genes. In some embodiments, the one or more mutated min gene are selected from the group consisting of a minC gene, a minD gene, and a combination thereof. In some embodiments, this is accomplished by deleting the min gene. Other approaches for mutating or otherwise decreasing expression of one or more min genes are contemplated to fall within the scope of the presently disclosed subject matter, such as but not limited to specific knock outs, targeted inactivations or excisions by any one of several approaches (exemplary, but not exclusively through CRISPR/Cas9, TALENS, ZFNs), knock downs, effects on promoters, conditional mutants and/or inducible mutants (e.g., for use in better growing up the bacteria that may be growth restricted by the mutations or gene inactivations), or in live attenuated bacterial vaccines.

In accordance with some embodiments of the presently disclosed subject matter, the terms “genome reduced”, “genome reduction”, or “GR” are used interchangeably and encompasses actual genomic deletions but also other modifications, such as inactivation, functional inactivation, and/or mutation, that reduce expression of one or more genes. In some embodiments, reducing and/or eliminating expression of genes in the bacteria yields enhanced immunogenicity, optionally enhanced immunogenicity of a peptide, polypeptide, or protein present on and/or in the bacterium and/or a derivative thereof (such as but not limited to a minicell produced therefrom). In some embodiments, the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes. In some embodiments, the reduced number of expressed genes comprises a reduction of expressed genes selected from the group consisting of at least about 2.4%, at least about 15.9%, and at least about 29.7%.

In some embodiments of the presently disclosed subject matter, there is a steady increase in immunogenicity as more and more genes are deleted, without a distinct “threshold effect” or notable discontinuity, which supports that beyond the effects of deleting a specific gene, there are effects due to the overall quantitative reduction in the number of genes.

Genes may be completely or partially deleted, for example by the methods employed by Hashimoto et al., 2005 and by the lambda Red systems described by Datsenko et al., 2000; by CRISPR/Cas9; and other methods to delete, inactivate, or decrease expression of bacteria genes, as would be apparent to one of ordinary skill in the art upon a review of the instant disclosure.

The presently disclosed subject matter relates in some embodiments to the effects on immunogenicity of expressing immunogens, such as vaccine antigens, in minicells from bacteria that have a reduced or eliminated expression of genes. Thus, in some embodiments, the minicell derived from the bacterium with fewer expressed genes and/or one or more mutated min genes is more immunogenic, as compared to an immune response that would have been induced in the subject by a minicell derived from a bacterium of the same strain that has a full complement of expressed genes and/or an unmutated min gene. In some embodiments, reducing and/or eliminating expression of one or more genes and/or mutating the min gene in the bacterium yields the enhanced immunogenicity. By way of example and not limitation, wholesale reduction of the bacterial genome, by small or large scale deletions, is one way this might be accomplished. Other approaches for decreasing expression of one or more genes fall within the scope of the presently disclosed subject matter, such as but not limited to specific knock outs, targeted inactivations or excisions by any one of several approaches (exemplary, but not exclusively through CRISPR/Cas9, TALENS, ZFNs), knock downs, effects on promoters, conditional mutants and/or inducible mutants (e.g., for use in better growing up the bacteria that may be growth restricted by the mutations or gene inactivations), or in live attenuated bacterial vaccines. In some representative, non-limiting embodiments, genes affecting surface structures can affected. Expression of protein structures can be affected, as can be non-protein structures.

In some embodiments, the minicell comprises a recombinant protein. In some embodiments, the recombinant protein is present in cytoplasm and/or on a surface of the minicell. In some embodiments, the recombinant protein is present in an enhanced amount as compared to an amount that would have been present in a minicell derived from a bacterium of the same strain that has a full complement of expressed genes and/or an unmutated min gene.

In some embodiments, the recombinant protein is an antigen, optionally an antigen on a surface of a membrane, or a derivative thereof. The antigen or immunogen is any antigen against which an immune response is desired. One or more such antigens can be provided by the minicell derived from the modified bacterium. Representative, non-limiting examples of antigens include an antigen to modulate autoimmune responses, an antigen for which it might be therapeutically useful to produce an immune response, such as fibrosis associated with atherosclerosis or the amyloid plaques of Alzheimer's disease or other degenerative diseases; an antigen used to induce an immune response against specific components of the immune system to modify autoimmune or allergic diseases; and/or combinations thereof.

The presently disclosed subject matter provides the following exemplary non-limiting embodiments.

Minicells derived from bacteria that have a reduced expression of a set of genes and that have an immunogen of interest, such as but not limited to on their surfaces, elicit an enhanced immune response against that immunogen compared to minicells from wild type, non-gene reduced bacteria.

In some embodiments, an expression vector comprising a nucleotide sequence encoding a protein of interest, such as a heterologous protein, such as an antigen, is provided. In some embodiments, the expression vector is configured to express the heterologous protein in a minicell derived from a modified bacterium of the presently disclosed subject matter. The presently disclosed subject matter encompasses any suitable expression vector as would be apparent to one of ordinary skill in the art upon a review of the instant disclosure. In some embodiments, the heterologous protein, such as an antigen, is expressed on the surface of the modified bacterium. In some embodiments, the vector comprises an autotransporter (AT) expression vector. In some embodiments, the vector comprises a codon optimized sequence encoding the antigen. In some embodiments, the AT expression vector comprises a monomeric vector or a trimeric vector. In some embodiments, the nucleotide sequence encoding the antigen is positioned under control of an inducible promoter or a constitutive promoter.

In some embodiments, the heterologous protein, such as an antigen, is expressed as a monomer or as a trimer. In some embodiments, the vector is provided in a pharmaceutically acceptable carrier. Thus, one way the heterologous protein, such as an antigen can be placed on the surface of the bacteria is using an autotransporter (including but not limited to a monomeric or trimeric embodiment), by itself, or in the context of a foreign protein or scaffold to enhance/improve formation of desired heterologous protein, such as an antigen. The autotransporter expression is not the only way to express a heterologous protein, such as an antigen. Heterologous proteins, such as an antigens, can be placed on the surface of the bacteria using other technologies, for example by covalent or non-covalent linkage, absorption, affinity tag, and the like. Using other technologies to place antigens or immunogens on the surfaces of the minicells derived from the reduced genome bacteria provides for the production of immunogens and/or vaccines directed against proteins or other antigens that cannot be expressed on the bacterial minicell surface using autotransporters or against non-protein antigens (such as but not limited to polysaccharides).

There are many other ways to express heterologous proteins, such as an antigens, and/or to specifically place them on the surfaces of the minicells, or even inside the bacterial minicell, such as but not limited to covalent coupling of the heterologous protein, such as an antigen, to the surface of the bacterial minicell, association of the bacterial minicell with heterologous protein, such as an antigen, non-covalently using an affinity tag, non-specific adsorption, and/or addition of a binding moiety to the heterologous protein, such as an antigen, followed by mixing the heterologous protein, such as an antigen, with the bacterial minicell.

The autotransporter expression cassette approach enables a synthetic biology solution: the protein (e.g., antigen) need not be isolated/purified/conjugated to carrier protein. Only the identity of the protein is needed. Then the coding sequence can be rapidly synthesized and cloned into the appropriate expression vector, followed by expression in the modified bacteria to provide for expression in the minicell.

The wild type/native protein can be used, or a component of the protein can be used, if it is desirable to produce an immune response only against a particular component of the protein. A mutated version of the protein can be used, to enhance immune responses or to bias immune responses (in a non-exclusive example, humoral vs. cellular), or direct immune responses toward a particular mutant version of the gene (for example, in a cancer application).

The antigen or immunogen, used interchangeably herein, can be used to elicit an immune response against a pathogen, as in developing a prophylactic vaccine. The immunogen can be used to elicit an immune response against a pathogen, as in developing a therapeutic vaccine, for example to treat a chronic infectious disease, including chronic viral diseases. One example would be HIV in an HIV-infected patient. TB is another application as are parasitic diseases.

The immunogen can be used to elicit an immune response against a non-pathogen, for example to manipulate the microbiome.

The immunogen can be used to elicit an immune response against a self-protein/proteins, as a way of modifying inflammatory or autoimmune diseases, for example by targeting particular cells or subsets of cells in the patient's immune system.

The immunogen can be used to elicit an immune response against a tumor antigen, as in developing a prophylactic vaccine against particular cancers in which tumor antigens are over-expressed, enhance tumor immune surveillance.

The immunogen can also be used to elicit an immune response against a tumor antigen, as in developing a therapeutic tumor vaccine.

The therapeutic tumor vaccine can be directed against known tumor antigens (native or modified for targeting or enhanced immunogenicity). In other words, stocks of premade tumor vaccines against know tumor antigens can be prepared. These could be used singly or in combination. They could be used by themselves, or along with treatments to enhance immune responses against those tumor antigens, such as immune checkpoint inhibitors.

The therapeutic tumor vaccine can be directed against tumor antigens identified on a custom basis. In other words, an individual patient's cancer can be studied, using techniques such as RNAseq, deep sequencing of the tumor DNA, and/or proteomics approaches, and then alone or in comparison to normal tissues from the same or other patients can be employed to select and design a sequence encoding the tumor antigen (native or modified). These could be used singly or in combination. The vaccines could be used by themselves, or along with treatments to enhance immune responses against those tumor antigens, such as but not limited to immune checkpoint inhibitors.

All of the above prophylactic and therapeutic uses can be in humans or animals. For example, the technology can be used to make veterinary prophylactic infectious disease vaccines.

The immunogen can be used to elicit the rapid production of antibodies in animals for the purposes of producing antibodies. These can be, for example, custom polyclonal antibodies, obtained directly from various species used to make custom polyclonal antibodies, such as rabbits, goats, sheep, horses, cows, and camelidae. The antibodies can be obtained from serum or from colostrum.

The immunogen can be used to immunize animals (e.g. mice, but also other species including but not limited to rabbits) to accelerate the production of monoclonal antibodies, since the first step in making a monoclonal antibody is to immunize an animal so that it makes antibodies, so that its spleen cells can be fused with myeloma cells to make a hybridoma. Such monoclonal antibodies can be used in all the analytic, diagnostic, and therapeutic ways in which monoclonal antibodies are typically used.

The bacterium can be any bacterium, including Gram-negative bacteria. E. coli are not the only genome reduced and/or one or more mutated min gene containing bacteria that can be used. Other Gram-negative bacteria can be used, and other genome reduced and/or one or more mutated min gene containing strains of other bacteria can be used, such as but not limited to genome reduced and/or one or more mutated min gene containing strains of Salmonella or even Vibrio. Such genome reduced and/or one or more mutated min gene containing versions of other bacterial species are prepared in accordance with techniques recognized in the art, as would be apparent to one of ordinary skill in the art up on a review of the instant disclosure, and then used to express immunogens such as but not limited to vaccine antigens. Thus, in some embodiments the bacteria are from Enterobacteriaceae, such as but not limited to Salmonella, Klebsiella, Shigella, Pseudomonas, Vibrio, Corynebacterium, and Yersinia. In some embodiments, representative bacteria can be chosen via a systematic review of the taxonomic tree; and thus, can include all Proteobacteria. Amino acid sequences for minC, minD, and minE gene products as well as for FtsZ gene products can be found in the GENBANK® biosequence database, and include but are not limited to those set forth herein above.

Minicells from bacteria modified as described herein, used by themselves without a recombinant antigen on their surfaces, can be used to elicit useful immune responses against those bacteria. Such minicells from bacteria modified as described herein can be used as prophylactic and/or therapeutic vaccines and/or to manipulate the composition of the microbiome.

Thus, in some embodiments, the presently disclosed subject matter relates to strategies for the rapid production of better immunogens for the production of new vaccines, including prophylactic vaccines for infectious diseases of humans and animals and therapeutic vaccines for cancer immunotherapy and/or for other diseases, disorders, and/or conditions where modulation of an immune response is therapeutically helpful. These strategies include:
1. Direct antigen production in a bacterial cell employing a synthetic biology approach in which the antigen of interest is expressed directly in the bacteria, directed by recombinant coding sequence. In some embodiments, antigens of interest are placed on the cell surface, and in some embodiments Gram-negative autotransporter protein expression cassettes are used to place antigens-of-interest on bacterial cell surfaces, e.g., minicell surfaces. Instead of purifying the protein antigen and conjugating it to carrier protein, antigen coding sequences can be cloned into an expression cassette. In the Gram-negative autotransporter embodiment discussed herein, these autotransporters (e.g., Type 5 Secretion Systems) place the antigen on the cell surface, e.g., minicell surface, as the vaccine immunogen. This obviates any need to isolate or synthesize the protein antigen, purify the antigen, couple the antigen to an appropriate carrier, and prepare a parental immunization, saving up to several weeks.
2. Use of genome reduced bacteria and/or one or more mutated min gene containing bacteria (such as but not limited to E. coli) to produce a minicell comprising the antigen. In some embodiments, the bacteria are Gram-negative bacteria, and in some embodiments the Gram-negative bacteria are E. coli. In representative embodiments, surface expressed antigen would be more accessible to the immune system and elicit better immune responses by expressing the antigens, such as but not limited to vaccine antigens, in minicells derived from genome reduced and/or one or more mutated min gene containing bacteria, in some embodiments on the surfaces of minicells derived from genome reduced and/or one or more mutated min gene containing bacteria, in some embodiments minicells derived from genome reduced and/or one or more mutated min gene containing Gram-negative bacteria, and in some embodiments on the surfaces of minicells derived from genome reduced and/or one or more mutated min gene containing E. coli.
3. Intranasal immunization. As a representative, non-limiting route of administration, intranasal immunization exposes M cells and dendritic cells directly to the immunogen, and the oropharyngeal mucosa has a large amount of lymphoid tissue, which produces enhanced immune responses to intranasally administered immunogens. However, the presently disclosed subject matter encompasses any route of administration as would be apparent to one of ordinary skill in the art upon a review of the instant disclosure, including but not limited to topical, oral, rectally, vaginally, intravenous, intramuscular, intra-arterial, intramedullary, intrathecal, intraventricular, transdermal, subcutaneous, intraperitoneal, enteral, sublingual, or in the case of a neoplasm, intratumorally.
4. Exponential increasing (exp-inc) immunization. In a representative, non-limiting embodiment, sequential, rapid exposure to increasing amounts of immunogen can yield enhanced immune responses, thought to occur because such immunogen exposure kinetics mimic the antigen exposure a host would experience in the face of a severe, poorly controlled infection, which would trigger an enhanced immune response. The immunogens discussed herein can be used in exp-inc immunization regimens to enhance immune responses against the antigen. Conversely, the immunogens described herein can be used in exponential decreasing dose administration patterns, or at repeated low doses to elicit a tolerizing response.

Embodiments of the presently disclosed subject matter relate at least in part to the use of minicells and genome reduced and/or one or more mutated min gene containing bacteria to produce an antigen capable of rapidly inducing an immune response against an antigen. The antigen-expressing minicells provide rapid antibody production for use in making custom polyclonal antibodies and materials needed (for example plasma cells) for monoclonal antibodies. The antigen-expressing minicells also can serve as vaccine immunogens designed to elicit immune responses that protect against infectious agents and/or vaccine immunogens designed to elicit a therapeutic immune response against a disease, disorder, or condition (e.g., cancers), and/or a therapeutic immune response designed to otherwise therapeutically modulate immune responses, for example in treatment autoimmune diseases.

As set forth herein, expressing an antigen in a minicell derived from a genome reduced and/or one or more mutated min gene containing bacterium can yield substantially higher binding of an antibody directed against the antigen to the minicell and that minicell comprising the test antigen can elicit a significantly higher immune response against the test antigen when an animal is immunized with a minicell comprising that test antigen than when immunized with a minicell derived from a wild type bacteria, and that minicell derived from that bacteria with progressively increasing amounts of genome deletion elicited increasingly potent immune responses.

Further, in some embodiments, an enhanced immune response in accordance with some embodiments of the presently disclosed subject matter involves enhanced cytotoxic T-cell responses directed against tumor and/or cancer cells and/or enhanced antibody responses directed against tumor and/or cancer antigens expressed in and/or on the surfaces of the tumor and/or cancer cells. Enhanced cytotoxic T-cell responses may be directed against previously identified tumor and/or cancer antigens or against newly identified antigens selected based on the analysis of genes or proteins differentially expressed in the tumor and/or cancer. Anti-tumor and/or cancer antibody responses may be directed against antigens conventionally targeted by monoclonal antibodies currently in use for anti-tumor and/or anti-cancer therapeutics and/or against novel antigens.

In some embodiments, a pharmaceutical composition comprising one or more components of the presently disclosed subject matter is administered orally. In some embodiments, it is administered intra-nasally, rectally, vaginally, parenterally, employing intradermal, subcutaneously, or intramuscularly. In some embodiments, the pharmaceutical composition is a vaccine.

The system can also be used to express viral proteins on the surface of minicells derived from these genome-reduced bacteria to be used for immunization or treatment directed against the viral proteins.

The presently disclosed subject matter provides a series of proteins or peptides and systems to produce or express those peptides in the context of cell structures, such as a lipid bilayer and other membrane structures found to have immunogenic activity that can be used singly or in combination to elicit an immunogenic response and are useful for preventing and treating infections associated with various viruses (such as HIV, SARS-COV, SARS-COV-2, MERS, etc.), and microbial infections (such as TB). The presently disclosed subject matter could also be used to produce immunizing antigens targeting the conserved regions of other virion envelope proteins, for or example, a universal influenza vaccine.

In some embodiments, the presently disclosed subject matter provides minicells derived from genome-reduced modified bacterium expressing a set of peptides that can be used together as a cocktail or individually as a component of a vaccine (immunogen) to prevent or to treat any condition, disease, and/or disorder as described herein. When administered, the minicell comprising the cocktail or combination of peptides elicits an immunogenic response. The presently disclosed subject matter further encompasses the use of biologically active homologues of the peptides and wells as biologically active fragments of the peptides. The homologues can, for example, comprise one of more conservative amino acid substitutions, additions, or deletions.

In some embodiments, the presently disclosed subject matter provides an immunogenic vaccine composition for use in treating and preventing viral infections and other microbial infections. In some embodiments, the composition comprises at least one isolated peptide selected from the group of peptides disclosed herein, or biologically active fragments or homologs thereof. In some embodiments, the immunogenic vaccine composition is a system comprising a viral peptide provided by a minicells derived from a bacterium in accordance with the presently disclosed subject matter. The vaccine composition can also include an adjuvant or a pharmaceutically acceptable carrier. In some embodiments, at least two peptides are included in the composition. Any combination of the peptides can be used.

In some embodiments, an immunogenic fragment or homolog of a peptide of the presently disclosed subject matter is used. In some embodiments, the biologically active fragments or homologs of the peptide share at least about 50% sequence identity with the peptide. In some embodiments, they share at least about 75% sequence identity with the peptide. In some embodiments, they share at least about 95% sequence identity with the peptide.

In some embodiments, at least one of the active fragments or homologs being used comprises a serine or alanine amino acid substitution for a cysteine residue. In some embodiments, at least one of the active fragments or homologs being used comprises at least one conservative amino acid substitution. The presently disclosed subject matter encompasses the use of amino acid substitutions at any of the positions, as long as the resulting peptide maintains the desired biologic activity of being immunogenic. The presently disclosed subject matter further includes the peptides where amino acids have been deleted or inserted, as long as the resulting peptide maintains the desired biologic activity of being immunogenic.

In some embodiments, the methods of the presently disclosed subject matter provide for administering the vaccine composition to a subject at least about 2 times to about 50 times. In some embodiments, the method comprises administering the vaccine composition to a subject at least about 5 times to about 30 times. In some embodiments, the methods of the presently disclosed subject matter provide for administering the vaccine composition to a subject at least about 10 times to about 20 times. The method also provides for administering the composition daily, or weekly, or monthly. One of ordinary skill in the art can design a regimen based on the needs of a subject, taking into account the age, sex, and health of the subject.

As described herein, the peptides provided by the minicells derived from the modified bacterium are immunogenic, so a useful composition comprising one or more of the peptides of the presently disclosed subject matter, even when using active fragments or homologs, or additionally short peptides, elicits an immunogenic response.

In some embodiments, a homolog of a peptide of the presently disclosed subject matter is one with one or more amino acid substitutions, deletions, or additions, and with the sequence identities described herein. In some embodiments, the substitution, deletion, or addition is conservative. In some embodiments, a serine or an alanine is substituted for a cysteine residue in a peptide of the presently disclosed subject matter.

In some embodiments, the subject is a mammal. In another embodiment, the mammal is a human.

The presently disclosed subject matter encompasses the use of purified isolated, recombinant, and synthetic peptides.

The presently disclosed subject matter further provides methods for producing peptides which are not easily soluble in an aqueous solution, by immediately expressing the peptides on the surface of the bacteria.

The methods and compositions of the presently disclosed subject matter encompass multiple regimens and dosages for administering the peptides of the presently disclosed subject matter for use in preventing and treating diseases and disorders caused by infectious agents. For example, a subject can be administered a combination of peptides, such as a combination of peptides provided by minicells derived from a bacterium, or a combination of bacteria expressing different peptides, of the presently disclosed subject matter once or more than once. The frequency and number of doses can vary based on many parameters, including the age, sex, and health of the subject. In some embodiments, up to 50 doses are administered. In some embodiments, up to 40 doses are administered, and in another up to 30 doses are administered. In some embodiments, up to 20 doses are administered, and in another up to 10 doses are administered. In some embodiments, 5-10 doses are administered. In some embodiments, 5, 6, 7, 8, 9, or 10 doses can be administered.

In some embodiments, minicells derived from bacteria expressing a peptide or bacteria expressing two or more peptides are administered more than once daily, in another daily, in another on alternating days, in another weekly, and in another, monthly. Treatment periods may be for a few days, or about a week, or about several weeks, or for several months. Follow-up administration or boosters can be used as well and the timing of that can be varied.

The amount of minicells derived from bacteria expressing a peptide or derivative of the bacteria administered per dose can vary as well. For example, in some embodiments, the compositions and methods of the presently disclosed subject matter include a range of peptide amounts (for example as provided by minicells derived from bacteria expressing a peptide) between about 1 nanogram of each peptide per dose to about 10 milligrams of immunogen per dose. In some embodiments, the number of micrograms is the same for each peptide. In some embodiments, the number of micrograms is not the same for each peptide. In some embodiments, the range of amounts of each immunogen administered per dose is from about 1 nanogram to about 10 milligrams.

Subjects can be monitored before and after minicell administration for antibody levels against the immunogens being administered (for example as provided by bacteria expressing a peptide) and by monitoring T cell responses, including CD4′ and CD8+. Methods for these tests are routinely used in the art and are either described herein or, for example, in publications cited herein.

Although a vaccine composition construct, minicells derived from bacteria or a mixture of bacteria, derivatives thereof, or cocktail of peptides or a combination therefor is described herein, when more than one bacterial construct or peptide is administered, each different bacterial construct or peptide can be administered separately. When a vaccine composition is administered more than once to a subject, the dose of each bacterial construct or peptide may vary per administration.

To increase the immunological response, various adjuvants may be used depending on the host species, including but not limited to cholera toxin B subunit, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as cholera toxin B subunit, alum, saponins, nucleic acids, LPS, BCG (Bacille Calmette-Guerin) and Corynebacterium parvum.

If peptides are to be placed on the minicells derived from the genome-reduced bacteria following exogenous production and not by protein synthesis by the bacteria themselves, those peptides for use in the presently disclosed subject matter may be readily prepared by standard, well-established techniques, such as solid-phase peptide synthesis (SPPS) as described by Stewart et al., 1984 and as described by Bodanszky & Bodanszky, 1984. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxycarbonyl as the α-amino protecting group, and the FMOC method which utilizes 9-fluorenylmethyloxycarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈-, or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Decarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and decarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tartaric, citric, benzoic, cinnamic, mandelic, methanesulfonic, ethanesulfonic, p-toluenesulfonic, salicylic and the like, to provide a water soluble salt of the peptide is suitable for use in the presently disclosed subject matter, for example a GR bacteria with attached additional immunogens.

The presently disclosed subject matter also provides for homologs of proteins and peptides for use in accordance with the presently disclosed subject matter. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, or more conservative amino acid changes typically have no effect on protein function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substantially pure protein or peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

One of ordinary skill in the art will appreciate that when more than one peptide is used (for example as provided by a minicell derived from bacterium expressing two or more peptides or by different bacteria expressing different peptides or derivative of the bacterium) that they do not necessarily have to be administered in the same pharmaceutical composition at the same time, and that multiple administrations can also be used. When multiple injections are used they can be administered, for example, in a short sequence such as one right after the other or they can be spaced out over predetermined periods of time, such as every 5 minutes, every 10 minutes, every 30 minutes, etc. Of course, administration can also be performed by administering a pharmaceutical comprising all components to be administered, such as a cocktail comprising a minicell derived from the bacteria expressing a peptide or derivative thereof. It can also be appreciated that a treatment regimen may include more than one round of injections, spaced over time such as weeks or months, and can be altered according to the effectiveness of the treatment on the particular subject being treated.

The presently disclosed subject matter provides multiple methods of using specifically prepared minicells derived from bacteria expressing a peptide or derivative thereof, for example, in fresh or lyophilized liposome, proper routes of administration of the minicell or derivative thereof, proper doses of the minicell or derivative thereof, and specific combinations of heterologous immunization including priming in one administration route followed by liposome-mediated antigen boost in a different route to tailor the immune responses in respects of enhancing cell mediated immune response, cytokine secretion, humoral immune response, especially skewing T helper responses to be Th1 or a balanced Th1 and Th2 type. For more detail, see U.S. patent application Ser. No. 11/572,453 (published as U.S. Patent Application Publication No. 2008/0193469 and incorporated herein by reference in its entirety), which claims priority to PCT International Patent Application Serial No. PCT/US2005/026102 (published as PCT International Patent Application Publication No. WO 2006/012539 and incorporated herein by reference in its entirety).

A homolog herein is understood to comprise an immunogenic peptide having in some embodiments at least 70%, in some embodiments at least 80%, in some embodiments at least 90%, in some embodiments at least 95%, in some embodiments at least 98%, and in some embodiments at least 99% amino acid sequence identity with the peptides mentioned above and is still capable of eliciting at least the immune response obtainable thereby. A homolog or analog may herein comprise substitutions, insertions, deletions, additional N- or C-terminal amino acids, and/or additional chemical moieties, such as carbohydrates, to increase stability, solubility, and immunogenicity.

In some embodiments of the presently disclosed subject matter, the present immunogenic polypeptides as defined herein, are glycosylated. Without wishing to be bound by any particular theory, it is hypothesized herein that by glycosylation of these polypeptides the immunogenicity thereof may be increased. Therefore, in some embodiments, the aforementioned immunogenic polypeptide as defined herein before, is glycosylated, having a carbohydrate content varying from 10-80 weight percent (wt %), based on the total weight of the glycoprotein or glycosylated polypeptide. Said carbohydrate content ranges can be from 15-70 wt %, or from 20-60 wt %. In another embodiment, said glycosylated immunogenic polypeptide comprises a glycosylation pattern that is similar to that of the peptides of the human that is treated. It is hypothesized that this even further increases the immunogenicity of said polypeptide. Thus, in some embodiments, the immunogenic polypeptide comprises a glycosylation pattern that is similar to that of the corresponding glycoprotein.

In some embodiments, the source of a peptide comprises an effective amount of at least one immunogenic peptide selected from the peptides described herein, and immunologically active homologs thereof and fragments thereof, or a nucleic acid sequence encoding said immunogenic peptide.

In some embodiments, the present method of immunization comprises the administration of a source of immunogenically active peptide fragments, said peptide fragments being selected from the peptide fragments and/or homologs thereof as defined herein before.

Peptides may advantageously be chemically synthesized and may optionally be (partially) overlapping and/or may also be ligated to other molecules, peptides, or proteins. Peptides may also be fused to form synthetic proteins, as in Welters et al., 2004. It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g. by lipidation or glycosylation. To enhance the solubility of the peptide, addition of charged or polar amino acids may be used, in order to enhance solubility and increase stability in vivo.

For immunization purposes, the aforementioned immunogenic peptides for use with the presently disclosed subject matter may also be fused with proteins, such as, but not limited to, tetanus toxin/toxoid, diphtheria toxin/toxoid or other carrier molecules. The polypeptides according to the presently disclosed subject matter may also be advantageously fused to heatshock proteins, such as recombinant endogenous (murine) gp96 (GRP94) as a carrier for immunodominant peptides as described in (see e.g., Rapp & Kaufmann, 2004; Zugel, 2001), or fusion proteins with Hsp70 (PCT International Patent Application Publication No. WO 1999/54464).

The individual amino acid residues of the present immunogenic (poly)peptides for use with the presently disclosed subject matter can be incorporated in the peptide by a peptide bond or peptide bond mimetic. A peptide bond mimetic of the presently disclosed subject matter includes peptide backbone modifications well known to those skilled in the art. Such modifications include modifications of the amide nitrogen, the alpha carbon, amide carbonyl, complete replacement of the amide bond, extensions, deletions, or backbone cross-links. See generally, Spatola, 1983. Several peptide backbone modifications are known and can be used in the practice of the presently disclosed subject matter.

Amino acid mimetics may also be incorporated in the polypeptides. An “amino acid mimetic” as used here is a moiety other than a naturally occurring amino acid that conformationally and functionally serves as a substitute for an amino acid in a polypeptide of the presently disclosed subject matter. Such a moiety serves as a substitute for an amino acid residue if it does not interfere with the ability of the peptide to elicit an immune response. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the presently disclosed subject matter are discussed by Morgan & Gainor, 1989.

In some embodiments, the present method comprises the administration of a composition (e.g., minicell or derivative thereof) comprising one or more of the present immunogenic peptides as defined herein above, and at least one excipient. Excipients are well known in the art of pharmacy and may for instance be found in textbooks such as Remington's Pharmaceutical Sciences, 18th ed. (1990).

The present method for immunization may further comprise the administration, and in some embodiments, the co-administration, of at least one adjuvant. Adjuvants may comprise any adjuvant known in the art of vaccination or composition for eliciting an immune response and may be selected using textbooks like Colligan et al., 1994-2004.

Adjuvants are herein intended to include any substance or compound that, when used, in combination with an antigen, to immunize a human or an animal, stimulates the immune system, thereby provoking, enhancing, or facilitating the immune response against the antigen, in some embodiments without generating a specific immune response to the adjuvant itself. In some embodiments, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant in some embodiments is capable of enhancing the immune response against at least two different antigens. The adjuvant of the presently disclosed subject matter will usually be a compound that is foreign to a human, thereby excluding immunostimulatory compounds that are endogenous to humans, such as e.g. interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637; referred to as nor-MDP), N-acetylmuramyl-L-alanyl-D-isoglutaminyl-L-alanine-2-(1′-2′-dip-almitoyl-sn-glycero-3-hydroxy-phosphoryloxy)-ethylamine (CGP 19835A, referred to as MTP-PE), DDA (2 dimethyldioctadecylammonium bromide), polyIC, Poly-A-poly-U, RIBI™, GERBU™, PAM3™, CARBOPOL™, SPECOL™, TITERMAX™, tetanus toxoid, diphtheria toxoid, meningococcal outer membrane proteins, cholera toxin B subunit, diphtheria protein CRM197. Exemplary, non-limiting adjuvants comprise a ligand that is recognized by a Toll-like-receptor (TLR) present on antigen presenting cells. Various ligands recognized by TLR's are known in the art and include e.g. lipopeptides (see e.g., PCT International Patent Application Publication No. WO 2004/110486), lipopolysaccharides, peptidoglycans, lipoteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications.

In some embodiments of the present methods, one or more minicell derived from bacteria expressing a peptide or derivative thereof are typically administered at a dosage of about 1 ug/kg patient body weight or more at least once. Often dosages are greater than 10 μg/kg. According to the presently disclosed subject matter, the dosages range in some embodiments from 1 μg/kg to 1 mg/kg.

In some embodiments typical dosage regimens comprise administering a dosage of in some embodiments 1-1000 μg/kg, in some embodiments 10-500 μg/kg, in some embodiments 10-150 μg/kg, once, twice, or three times a week for a period of one, two, three, four or five weeks. According to some embodiments, 10-100 μg/kg is administered once a week for a period of one or two weeks.

The presently disclosed methods, in some embodiments, comprise administration of one or more minicell derived from bacteria expressing a peptide or derivative of the minicell and compositions comprising them via the injection, transdermal, intranasal, or oral route. In some embodiments of the presently disclosed subject matter, the present method comprises vaginal or rectal administration of the present minicell derived from bacteria expressing a peptide or derivative of the minicell and compositions comprising them.

In some embodiments, of the presently disclosed subject matter relates to a pharmaceutical preparation comprising as the active ingredient the present source of a polypeptide as defined herein before. More particularly pharmaceutical preparation comprises as the active ingredient one or more of the aforementioned immunogenic peptides, homologues thereof and fragments of said peptides and homologs thereof, as provided by minicell derived from bacteria expressing a peptide or derivative of the minicell as defined herein above.

The presently disclosed subject matter further provides a pharmaceutical preparation comprising minicell derived from bacteria expressing a peptide or derivative of the minicell of the presently disclosed subject matter. The concentration of said peptides in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic peptide or bacteria expressing a peptide or derivative of the bacteria to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In some embodiments, the present minicell derived from bacteria expressing a peptide or derivative of the minicell are administered by injection. The parenteral route for administration is in accordance with known methods, e.g., injection or infusion by intravenous, intraperitoneal, intramuscular, intra-arterial, subcutaneous, rectal, vaginal, or intralesional routes. minicell derived from bacteria expressing a peptide or derivative of the minicell may be administered continuously by infusion or by bolus injection. In some embodiments, a composition for intravenous infusion could be made up to contain 10 to 50 ml of sterile 0.9% NaCl or 5% glucose optionally supplemented with a 20% albumin solution and in some embodiments between 10 μg and 50 mg, in some embodiments between 50 μg and 10 mg, of the minicell derived from bacteria expressing a peptide or derivative of the minicell. A typical pharmaceutical composition for intramuscular injection would be made up to contain, for example, 1-10 ml of sterile buffered water and in some embodiments between 10 μg and 50 mg, in some embodiments between 50 μg and 10 mg, of the minicell derived from bacteria expressing a peptide or derivative of the minicell of the presently disclosed subject matter. Methods for preparing parenterally administrable compositions are well known in the art and described in more detail in various sources, including, for example, Remington's Pharmaceutical Sciences, 18th ed. (1990), incorporated by reference in its entirety for all purposes).

For convenience, immune responses are often described in the presently disclosed subject matter as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of the peptides of the presently disclosed subject matter.

In certain embodiments, the disclosed methods and compositions may involve preparing peptides with one or more substituted amino acid residues. In various embodiments, the structural, physical, and/or therapeutic characteristics of peptide sequences may be optimized by replacing one or more amino acid residues.

In some embodiments, the presently disclosed subject matter encompasses the substitution of a serine or an alanine residue for a cysteine residue in a peptide of the presently disclosed subject matter. Support for this includes what is known in the art. For example, see the following citation for justification of such a serine or alanine substitution: Kittlesen et al., 1998.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C₁-C₁₀ carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, amino acids for which the hydropathic indices are in some embodiments within +/−2, in some embodiments within +/−1, and in some embodiments within +/−0.5 are employed.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In some embodiments, amino acids are replaced with others of similar hydrophilicity.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e.g., PROWL Rockefeller University website. For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, Mclachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

The presently disclosed subject matter is also directed to methods of administering the compounds of the presently disclosed subject matter to a subject.

Pharmaceutical compositions comprising the present compositions are administered to an individual in need thereof by any number of routes including, but not limited to, topical, oral, rectally, vaginally, intravenous, intramuscular, intra-arterial, intramedullary, intrathecal, intraventricular, transdermal, subcutaneous, intraperitoneal, intranasal, enteral, topical, sublingual, or rectal means.

The presently disclosed subject matter is also directed to pharmaceutical compositions comprising the minicells of the presently disclosed subject matter. More particularly, such compounds can be formulated as pharmaceutical compositions using standard pharmaceutically acceptable carriers, fillers, solubilizing agents, and stabilizers known to those skilled in the art.

The presently disclosed subject matter also encompasses the use of pharmaceutical compositions of an appropriate compound, homolog, fragment, analog, or derivative thereof to practice the methods of the presently disclosed subject matter, the composition comprising at least one appropriate compound, homolog, fragment, analog, or derivative thereof and a pharmaceutically-acceptable carrier.

The pharmaceutical compositions useful for practicing the presently disclosed subject matter may be administered to deliver a dose of between 1 ng/kg/day and 100 mg/kg/day. Pharmaceutical compositions that are useful in the methods of the presently disclosed subject matter may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the presently disclosed subject matter.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the presently disclosed subject matter may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology. A formulation of a pharmaceutical composition of the presently disclosed subject matter suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

Liquid formulations of a pharmaceutical composition of the presently disclosed subject matter which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the presently disclosed subject matter may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the presently disclosed subject matter may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the presently disclosed subject matter may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the presently disclosed subject matter may also be prepared, packaged, or sold in a formulation suitable for rectal administration, vaginal administration, parenteral administration

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example.

Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the presently disclosed subject matter may be prepared, packaged, or sold in a formulation suitable for pulmonary administration via the buccal cavity. Such a formulation may comprise dry particles which comprise the active ingredient and which have a diameter in the range from in some embodiments about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container.

In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. Dry powder compositions in some embodiments include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the presently disclosed subject matter formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration in some embodiments have an average diameter in the range from about 0.1 to about 200 nanometers.

The formulations described herein as being useful for pulmonary delivery are also useful for intranasal delivery of a pharmaceutical composition of the presently disclosed subject matter.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the presently disclosed subject matter may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, have in some embodiments an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Remington's Pharmaceutical Sciences, 18th ed. (1990), which is incorporated herein by reference.

Typically, dosages of the composition of the presently disclosed subject matter which may be administered to an animal, in some embodiments a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. In some embodiments, the dosage of the compound will vary from about 10 μg to about 10 g per kilogram of body weight of the animal. In another embodiment, the dosage will vary from about 10 mg to about 1 g per kilogram of body weight of the subject.

The composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the sex and age of the subject, etc.

The presently disclosed subject matter further provides kits comprising minicell derived from bacteria expressing a peptide or derivative of the minicell of the presently disclosed subject matter useful for eliciting an immunogenic response, and further includes an applicator and an instructional material for the use thereof.

### IV. Other Embodiments

In some embodiments, the presently disclosed subject matter also provides other systems by which antigens and/or immunogens of interest can be expressed in, on the surface of, or otherwise by bacteria. Thus, it is understood that the autotransporter expression system described herein is not the only way to express antigens. There are many other ways to express antigens and to specifically place them on the surfaces of the bacteria and/or minicells derived therefrom, or even inside the bacteria and/or minicells derived therefrom.

Similarly, the presently disclosed subject matter also provides modified bacteria other than modified E. coli. By way of example and not limitation, other Gram-negative bacteria can be used, and other genome reduced and/or one or more mutated min gene containing strains of other bacteria can be used, such as but not limited to genome reduced and/or one or more mutated min gene containing strains of Salmonella or even Vibrio. Such genome reduced and/or one or more mutated min gene containing versions of other bacterial species are prepared in accordance with techniques recognized in the art, as would be apparent to one of ordinary skill in the art up on a review of the instant disclosure, and then used to express immunogens such as but not limited to vaccine antigens. Thus, in some embodiments the bacteria are from Enterobacteriaceae, such as but not limited to Salmonella, Klebsiella, Shigella, Pseudomonas, Vibrio, Corynebacterium, and Yersinia. In some embodiments, representative bacteria can be chosen via a systematic review of the taxonomic tree: and thus, can include all Proteobacteria. Amino acid sequences for minC, minD, and minE gene products as well as for FtsZ gene products can be found in the GENBANK® biosequence database, and include but are not limited to those set forth herein above. As such, one of ordinary skill in the art could employ the present disclosure as a guide to construct genome reduced versions of other bacterial species and/or bacteria with min gene mutations (e.g., minC and/or minD mutations) and/or bacteria that overexpress FtsZ gene products for use in preparing minicells, including minicells that express vaccine antigens.

Furthermore, whereas in some embodiments the presently disclosed subject matter relates to the rapid production of antibodies, the presently disclosed subject matter also relates in some embodiments to the production of prophylactic vaccines for infectious diseases and/or therapeutic vaccines for infectious diseases (such as but not limited to chronic infectious diseases like HIV, other chronic viral diseases, TB, and/or parasitic diseases), therapeutic vaccines for cancer (e.g., off the shelf vaccines directed at know cancer antigens) and custom vaccines designed based on the analysis of the cancer neoantigens for a given patient's cancer (i.e., a personalized anti-cancer vaccine), and therapeutic vaccines for other diseases, particularly diseases involving inflammatory processes, like autoimmune diseases, fibrosis, atherosclerosis, etc.

The antigen can be any desired antigen as would be apparent to one of ordinary skill in the art upon a review of the instant disclosure. In some embodiments, the antigen is derived from a microbe. In some embodiments, the antigen is derived from a cancer. In some embodiments the antigen is derived from a host protein that mediates other diseases or undesirable phenotypes, including in some embodiments autoimmune or inflammatory diseases, or diseases in which the expression of a particular host protein mediates a disease process. In some embodiments, the antigen is derived from a cancer, or a target of an inappropriate or undesirable immune response, or a component of the host immune system, such as (but not exclusively), a host immune system component that, when targeted for destruction or inactivation or activation, alter an undesirable immune response.

In some embodiments, a method for producing an antibody or a desired cell-mediated immune response in a subject is disclosed. In some embodiments, the method comprises providing a minicell derived from bacteria expressing a peptide or derivative of the minicell in accordance with the presently disclosed subject matter and administering the minicell derived from bacteria expressing a peptide or derivative of the minicell to a subject in an amount and via a route sufficient to produce an antibody or a desired cell-mediated immune response in the subject against the antigen expressed by the minicell derived from bacteria expressing a peptide or derivative of the minicell or against cells expressing the antigen. Optionally, the production of the antibody or cell mediated immune response is enhanced in the subject as compared to an immune response produced in a subject by a minicell derived from a bacterium of the same strain that has a full complement of expressed genes and that expresses the antigen on its surface. In some embodiments, the administering of the modified bacterium to the subject is intranasally, transmucosally, including but not limited to orally, rectally, and vaginally; subcutaneously, intradermally, intramuscularly, other parenteral routes, or any combination thereof.

In some embodiments, a method for vaccinating a subject in need thereof is provided. In some embodiments, the method comprises providing a vaccine composition of the presently disclosed subject matter and administering the vaccine composition to the subject. In some embodiments, a method for treating a cancer or inappropriate immune responses or expression or production of a deleterious material in a subject in need thereof is provided, the method comprising providing a vaccine composition according to the presently disclosed subject matter and administering the vaccine to the subject. In some embodiments, a method for treating a cancer in a subject in need thereof is provided. In some embodiments, the inappropriate immune response or expression or production of a deleterious material is an autoimmune process, a method for altering the production or expressing of a pathogenic protein, and/or modifying or attacking or killing cells mediating disease. In some embodiments, the vaccine composition is administered orally, rectally, vaginally, intra-nasally, parenterally, intradermally, subcutaneously, or intramuscularly.

In some embodiments, the presently disclosed subject matter provides cancer antigens to immunize the endogenous immune system, i.e., “vaccinating” the subject against their own cancer. In some embodiments, the cancer is a drug resistant cancer or drug sensitive cancer. In some embodiments, the cancer is a cancer characterized by the presence of or as a solid tumor or liquid tumor, or is a cancer of hematologic origin. Thus, in some embodiments, the cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Bacteria, minC and minD mutants. The deleted bacterial strains with different percentages of the bacterial genome deleted were the kind gift of J. Kato (Hashimoto et al., 2005; Kato & Hashimoto, 2007), Tokyo Metropolitan University, Tokyo, Japan. The National Bioresource Project, E. coli Strain Office, National Institute of Genetics, Japan, provided the E. coli strains used in this study, derivatives of MG1655, include ME 5000 (with 0% of the genome deleted-reference wild type), ME 5010 (2.4% deleted), ME 5119 (15.8% deleted) and ME 5125 (29.7% deleted). Strains were grown in Luria-Bertani (LB) media and on LB agar plates, with the appropriate antibiotics as needed.

Electrocompetent cells and transformation with plasmid vectors. Bacteria were grown overnight at 37° C., shaking at 210 rpm in LB broth supplemented with selective antibiotic dependent strain specific resistance (streptomycin or ampicillin/chloramphenicol). New LB media cultures were inoculated from the overnight cultures and grown to log phase (OD₆₀₀˜0.4) with selective antibiotic. Due to their slower doubling times, the ME 5125 strains required an additional day of growth in a new secondary inoculum between the primary inoculum and the third inoculum grown to log phase. The cells were centrifuged at 1,000×g for 20 minutes at 4° C. and washed with sterile ice-cold H₂O+10% glycerol. They were transformed by electroporation using the Gene Pulser Xcell electroporation system (Bio-Rad Laboratories, Inc., Hercules, California, United States of America) with pRAIDA2, or pMP2463 (Stuurman et al., 2000). Electroporation was conducted in 0.1 cm electroporation cuvettes (Bio-Rad) with the following settings: 1800 V, 25 μF, 200Ω (Anthony, 2003). The electroporated cells were immediately transferred to 2 mL microfuge tubes with 1 mL of SOC media (Invitrogen brand, Thermo Fisher Scientific Inc., Waltham, Massachusetts, United States of America), grown at 37° C., 80 rpm for 1 hour, and plated on LB agar plates containing 50 μg/mL kanamycin.

Rhamnose induction. Bacteria transformed with pRAIDA2 were grown overnight in LB broth and 50 μg/mL kanamycin at 37° C., 210 rpm. The next day, new cultures were diluted to an OD₆₀₀ of 0.1 in fresh LB broth and 50 μg/mL kanamycin, then incubated at 37° C., 210 rpm until the OD₆₀₀ reached 0.4-0.7. GR ME 5125 strains required an additional day of growth in a new secondary inoculum between the primary inoculum and the final inoculum grown to log phase (OD₆₀₀˜0.4). Bacterial cultures were induced with 5 mM of rhamnose for 2 hours. The OD₆₀₀ of each culture was measured and aliquots of bacteria from each culture was saved for analysis by immunoblot. Afterwards, all cultures were centrifuged at 3,000×g for 10 minutes at 4° C. and pellets were stored in −20° C. for ImageStream analysis.

The minC and minD genes were deleted using a modification of the Lambda Red recombination methodology (Datsenko & Wanner, 2000). Briefly, the different E. coli strains were made electrocompetent by standard procedures (see e.g., Green & Sambrook, 2012). The cells were then transformed with pKD46, the plasmid having a thermosensitive origin of replication expressing the recombination machinery components (proteins Gam, Bet, and Exo) by electroporation using the Eppendorf Multiporator 4308, employing settings U=2500 V, tau=5 ms. Bacteria were then propagated at 30° C. We then produced a kanamycin resistance expression fragment for transformation and recombination with minC minD homology regions by PCR amplification using the pKD4 plasmid which includes the kanamycin resistance gene (Datsenko & Wanner, 2000) as template. The PCR primers were: 5′-GTGACTTGCCTCAATATAATCCAGACTATAACATGCCTTATAGTCTTCGGAACATC ATCGCGCGCTGGCGATGATTAATAGCTAATTGAGTAAGGCCAGGGTGTAGGCTGG AGCTGCTTC-3′ (SEQ ID NO: 7) and 5′-CGCTGCGACGGCGTTCAGCAACAATAA TCTGCAGCCGTTCTTTTGCAATGTTGGCTGTGTTTTTCTTCCGCGAGAGAAAGAAA TCGAGTAATGCCATAACATGGGAATTAGCCATGGTCC-3′ (SEQ ID NO: 8).

The PCR reaction was performed using an Eppendorf Mastercycler Gradient 5331 (Eppendorf, Germany) with the following conditions: 95° C. for 3 minutes; followed by 35 cycles of 95° C. for 30 seconds; 57.5° C. for 30 seconds; and 72° C. for 2 minutes; followed by 72° C. for 10 minutes; and then held at 4° C. PCR-amplified fragments were introduced into bacterial cells by electroporation using the Eppendorf Multiporator 4308, employing settings U=2500 V, tau=5 ms. After selection on kanamycin plates at 37° C., bacterial colonies were screened by PCR using primers 5′-GATTGAACAAGATGGATTGCACGC-3′ (SEQ ID NO: 9) and 5′-CTCGTCAAGAAGGCGATAGAAGGC-3′ (SEQ ID NO: 10), using the Eppendorf Mastercycler Gradient 5331 with the following conditions: 95° C. for 2 minutes 30 seconds; followed by 33 cycles of 95° C. for 30 seconds; 57° C. for 30 seconds; and 72° C. for 1 minute 40 seconds; followed by 72° C. for 10 minutes; and then samples were held at 4° C. PCR products were analyzed by agarose gel electrophoresis. The recombination event was verified by sequencing (Eurofins) to confirm replacement of minC and minD genes by the kanamycin resistance cassette. Bacteria were cured of the temperature sensitive origin of replication pKD46 plasmid by serial passage at 37° C. as described in Datsenko & Wanner, 2000. Microscopic examination, dynamic light scattering, and image cytometry further confirmed the acquisition of the minicell production phenotype (see below). The minC minD mutant bacteria were then cured of kanamycin resistance by transformation with a FLP recombinase-expressing gentamycin-resistant modification of plasmid pCP20 (Doublet et al., 2008; kind gift of B. Doublet, INRA, Infectiologie Animale et Santé Publique, Nouzilly, France) as described in Datsenko & Wanner, 2000 and as modified as per Barrick et al., 2017. Individual colonies were picked and propagated overnight in the liquid culture at 43° C. to induce recombination and loss of the pCP20-derivative plasmid. Restoration of a kanamycin-sensitive phenotype was verified by plating on kanamycin LB agar plates and sequenced to confirm removal of the kanamycin resistance gene. The minicell production phenotype was reconfirmed.

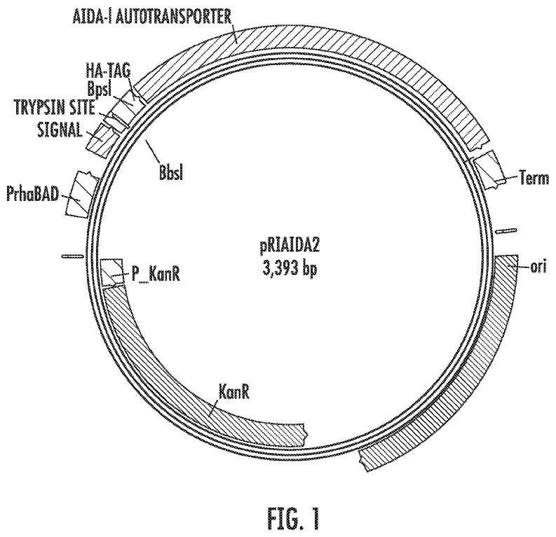

Expression plasmids for evaluation of minicell production and expression of recombinant proteins. The green fluorescent protein (GFP)-expressing plasmid, pMP2463 (Stuurman et al., 2000), was obtained from addgene.com (https://www.addgene.org/107774/). In preliminary experiments, it was determined that there was sufficient baseline expression of GFP from this plasmid. This enabled detection of GFP and evaluation of enrichment of GFP in minicells in the system under the growth and culture conditions used without further induction. We commissioned the synthesis of pRAIDA2 (see FIG. 1; Genesys). pRAIDA2 includes an expression cassette based on the AIDA-I (Benz & Schmidt, 1989) autotransporter (Jose & Meyer, 2007; Benz & Schmidt, 2011) under the control of a rhamnose-inducible promoter, an origin of replication, and a kanamycin resistance gene (Maeda et al., 2021). The parental version of pRAIDA2 expresses an influenza HA immunotag on a stuffer fragment within the expression cassette's cloning site. The stuffer fragment is flanked by Bbs I, Type IIS restriction sites, to enable cloning of synthetic DNAs into the plasmid to enable surface expression of proteins of interest. The sequence of pRAIDA2 has been deposited into the GENBANK® resource with Accession Number MW383928 (SEQ ID NO: 11). Plasmids were prepared using Qiagen Plasmid Mini Prep kit, quantitated, and assessed for quality spectrophotometrically.

Expression plasmid transformation. To prepare competent cells, bacteria were grown overnight at 37° C., shaking, in LB broth. LB broth was inoculated from overnights and grown to log phase. Cells were made electrocompetent as described above and transformed via electroporation with pMP2463 or pRAIDA2. Electroporation was conducted in 0.1 cm electroporation cuvettes with the Gene Pulser Xcell electroporation system (Bio-Rad) and pulsed at the following settings: 1800 V, 25 μF, and 200Ω. Electroporated cells were immediately transferred to 10 ml tubes with 1 mL of SOC media (Life Technologies), and grown at 37° C. for 1 hour before plating on LB agar plates containing the appropriate antibiotic.

Minicell purification and characterization. Minicells were produced as described in Lee al., 2015 with modifications. A 3-5 ml miniprep culture of engineered bacteria was initiated from a glycerol stock or from a colony grown on a fresh plate. The culture was grown at 37° C. for 6-8 hours or overnight (in the case of ME 5125 strain) and expanded to 50 ml culture growing overnight at 37° C. The next morning, this culture was used to start a 1-2 liter culture which was grown for 20-24 hours at 37° C. with half of the usual antibiotic concentration. If required, the surface expression of the HA epitope was induced in the cells transformed with pRAIDA2 with rhamnose at 0.5 mM added at the start of 1 liter minicells maxiprep.

Bacteria were separated from minicells by centrifugation for 10 minutes at 4000×g; supernatant was subjected to 10000×g, 12 minute centrifugation to sediment minicells. The minicell pellet was resuspended in 10-15 ml PBS and subjected to two subsequent 3000×g, 10 minute centrifugations to pellet residual bacteria. As the final step, to wash minicells from bacterial growth media, their volume was increased to 50 ml and minicells were spun down at 10000×g, 25 minutes at 15° C. The final minicell pellet was resuspended in PBS and stored at −20° C. The characteristics and quality of the minicell preparations were verified by dynamic light scattering and cryo-EM.

Electron microscopy. The size and morphology of minicells were measured using a FEI Tecnai F20 (FEI, Hillsboro, Oregon, United States of America) transmission electron microscope operating at 120 kV cryo-Electron microscopy (cryo-EM). The cryo-EM samples were prepared by a standard vitrification method. An aliquot of ˜3 μl sample solution was applied onto a glow-discharged perforated carbon-coated grid, (2/1-3C C-Flat; Protochips, Raleigh, North Carolina, United States of America) and the excess solution was blotted with filter paper. The samples were then quickly plunged into a reservoir of liquid ethane at −180 C. The vitrified samples were stored in liquid nitrogen and transferred to a Gatan 626 cryogenic sample holder (Gatan, Pleasantville, California, United States of America) and then maintained in the microscope at −180° C. All images were recorded with a Gatan 4K×4K pixel CCD camera under cryo-condition at a magnification of 9600× or 29,000× with a pixel size of 1.12 nm or 0.37 nm, respectively, at the specimen level, and at a nominal defocus ranging from −1 to −3 μm. The unfiltered samples were recorded at 9600×, images were recorded with a 500 nM magnification bar, filtered samples were recorded at 29,000×, and images were recorded with a t 100 nM magnification bar.

Immunoblots. Purified minicells and bacteria in the amount of 100-400 μg of protein were centrifuged for 15 minutes at 13-14000×g at 10° C. Pellets were resuspended in NuPAGE 1×LDS electrophoretic sample buffer (Invitrogen) containing 3%2-mercaptoethanol and heated at 100° C. for 5 minutes. Samples in the amount 50-100 μg (GFP) or 200-400 μg (HA tag) were separated using NuPAGE 4-12% precast gels (Life Technologies) in SDS-containing buffer and transferred to an Immobilon membrane (Bio-Rad). After 15 minute blocking in 1% casein blocker in TBS (ThermoFisher Scientific 37532), membranes were incubated with the primary antibody overnight at 4° C. (Abcam 183734, at 1:3000 dilution), washed, and incubated with a horseradish peroxidase-conjugated secondary antibody. Primary and secondary antibodies were diluted in 0.05% TBS/Tween-20 containing 0.05% casein blocker. Protein bands were visualized using a chemiluminescence kit (ThermoScientific). Images were taken by ChemiXX6 G:box digital imaging system (Syngene) and quantified using GeneTools software (Syngene). For quantitation, within each condition, to determine the enrichment of GFP or HA in the minicells, the signal due to GFP or HA was normalized to the signal due to the Dnak internal quantitation standard.

Image Cytometry. Image cytometry assays were acquired on an ImagestreamX MKII (Luminex) using 488 nm and 785 nm Scatter lasers at 60× magnification to check (a) expression of green fluorescent protein in cytoplasm of bacteria transformed with pMP2463; (b) expression of HA-immunotag on outer membrane of bacteria transformed with pRAIDA2; and (c) cell morphology and size. Pellets stored in −20° C., after rhamnose induction, were thawed and formalin-fixed (1×HBSS+0.2% formalin) and incubated at 37° C., 180 rpm for 1 hour. 5×10⁷ formalin fixed and washed cells were blocked with 1×PBS+10% FBS on ice for 20 minutes, then washed and centrifuged at 800×g with 1×PBS+2% FBS for 5 minutes at 4° C. Cells were incubated with 1:200 dilution of HA tag monoclonal antibody (Invitrogen) for 30 minutes at 4° C. Cells were washed two times and centrifuged at 800×g with 1×PBS+2% FBS for 5 minutes at 4° C. Cells were incubated with 1:600 dilution of goat anti-mouse IgG Alexa488 (ThermoFisher) for 30 minutes at 4° C. After incubation, cells were washed two times and centrifuged at 800×g with 1×PBS+2% FBS for 5 minutes at 4° C. then resuspended wash buffer and transferred into a 96 well flat bottom plate and stored at 4° C. until analyzed by ImagestreamX MKII (Luminex).

The pellet of cells transformed with either pRAIDA2 (HA-expressing) or pMP2463 (GFP-expressing) were thawed post-rhamnose induction, formalin-fixed (1×HBSS+0.2% formalin) and then incubated at 37° C., 180 rpm for 1 hour. Cells were washed with 1×PBS and centrifuged at 3,000×g for 6 minutes at 4° C. and then resuspended in 5 mL of 1×PBS before measuring the OD₆₀₀ nm. After calculations, 100 μl of 5×10⁷ were aliquoted into the wells of a 96 well flat bottom plate and stored at 4° C. until analyzed by ImagestreamX MKII (Luminex).

Eppendorf tubes containing minicells were spun down at 15,000-17,000×g for 15 minutes at 4° C. and supernatant was discarded. Pellets were resuspended in 1 mL of fixation solution (HBSS+0.2% formalin) and incubated at 37° C., 180 rpm for 1 hour. 5×10⁷ formalin fixed minicells were washed and centrifuged at 10,000×g for 12 minutes at 4° C. before being blocked with 1×PBS+10% FBS on ice for 20 minutes. The minicells were then washed and centrifuged at 800×g with 1×PBS+2% FBS for 4 minutes at 4° C. For minicells made from bacteria transformed with pRAIDA2, minicells were incubated with 1:200 dilution of anti-HA immunotag monoclonal antibody (Invitrogen) for 30 minutes at 4° C. Cells were washed two times and centrifuged at 800×g with 1×PBS+2% FBS for 5 minutes at 4° C. Cells were incubated with 1:600 dilution of goat anti-mouse IgG Alexa488 (ThermoFisher) for 30 minutes at 4° C. Post incubation, cells were washed two times and centrifuged at 800×g with 1×PBS+2% FBS for 5 minutes at 4° C. then resuspended wash buffer and transferred into a 96 well flat bottom plate and stored at 4° C. until analyzed by ImagestreamX MKII (Luminex).

Imagestream acquisition was set to collect 20,000 bacteria per sample. Objects were analyzed using IDEAS software 6.2.64.0. Focused bacteria were gated using the Gradient_RMS parameter for the brightfield channel. Single bacteria were then gated using the Aspect Ratio and Area parameters. Minicells were gated by Scatter and GFP/Alexa 488 signal intensity to distinguish whole rod, shaped bacteria from minicells.

For quantitation by the Amnis ImageStream instrument of minicell size, area measurements of histograms was determined via the ImageStream MKII. The area in square micrometers is calculated from the number of pixels with 1 pixel=0.25 μm². Area was then plotted as histograms using IDEAS 6.2 analysis software and FCS Express 7.

Dynamic Light Scattering (DLS). DLS measurements were performed on the isolated minicells. Size distribution of minicells resuspended in PBS was analyzed by dynamic light scattering using Zetasizer (Malvern Instruments model ZEN 3690, Malvern, Worcestershire, WR141XZ, United Kingdom). Percentage of particles of a specific size vs particle diameter was measured three times at 25° C. A representative graph is shown for each measurement. The original screen-shot captured image from the DLS instrument was overtraced using Affinity Designer to enhance legibility by reducing the pixilation of the captured image and to limit the data output range to the informative size region (100-10,000 nm).

Statistical and data analysis. Data were analyzed with SAS 9.4 and R (v1.3.1093) with the Rstudio environment with included packages, and the tidyverse and stats packages, with visualization using ggplot2. The Western blot experiments were analyzed as randomized block experiments, using two-way ANOVA with no interaction. Data were transformed to the log scale to better meet the assumptions underlying ANOVA and to facilitate interpretations as fold change.

### Example 1

Production of the minC/minD Mutations in Genome Reduced E. coli Strains

To explore whether it is possible to mutate the minC and minD genes of GR bacteria and then make minicells from those mutant bacteria, the comprehensive collection of genome deletions produced by the Tokyo Metropolitan University Group (Hashimoto et al., 2005; Kato & Hashimoto, 2007) from a derivative of E. coli MG1655 was obtained. The following strains were employed: ME 5000 (parental, non-deleted strain), ME 5010 (2.4% deleted), ME 5119 (15.8% deleted) and ME 5125 (29.7% deleted, the most deleted strain in the collection). These strains, while they are alive, grow with a significantly reduced doubling time (˜40 min for the 29.7% deleted strain), and altered morphology. Lambda Red recombineering (Datsenko & Wanner, 2000) was employed to delete the minC and minD genes. The presence of the inserted marker was screened for, and the was confirmed mutation by colony PCR and sequencing. The strain was then cured of kanamycin resistance by recombination with a derivative of pCP20 (Datsenko & Wanner, 2000; Doublet et al., 2008), and the removal of the kanamycin resistance gene was verified phenotypically, confirmed removal with sequencing, and reconfirmed the minicell production phenotype as described below. To examine expression in the minicells made from the genome reduced bacteria, plasmid pMP2463, which expresses GFP, was employed, and the synthesis of a plasmid, pRAIDA2 (see FIG. 1), which includes an AIDA-I-based autotransporter expression cassette (Benz & Schmidt, 1989; Maeda et al., 2021) was generated. In its parental form, pRAIDA2 has an influenza HA immunotag in the autotransporter expression cassette.

### Example 2

### Characterization of the Minicell Production Phenotype

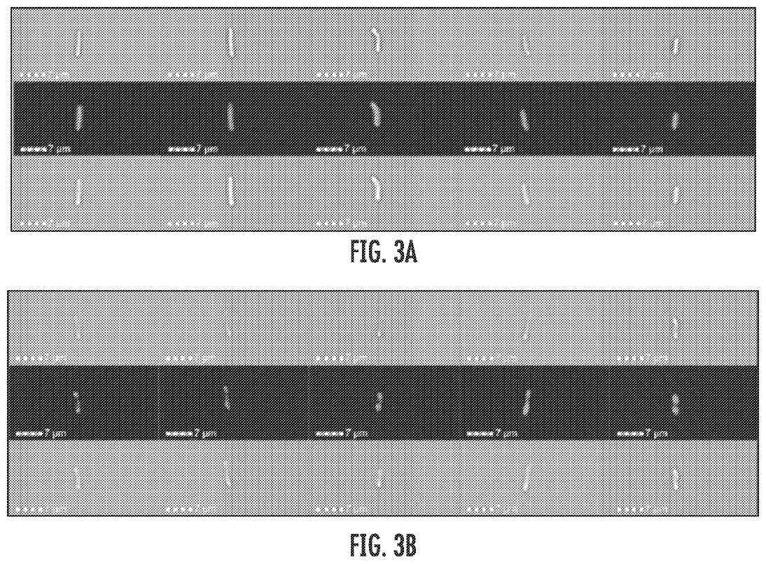

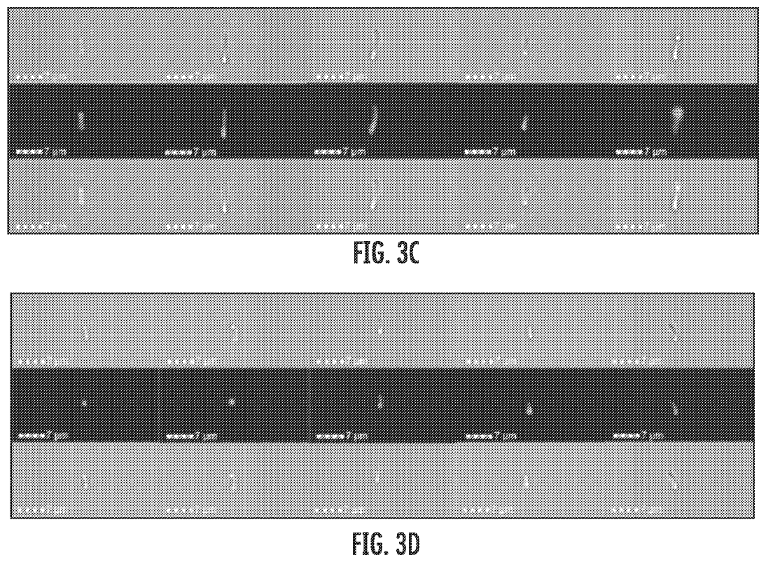

A comparison of the non-deleted ME 5000 strain and the 29.7% deleted ME 5125 strain is described herein. Image cytometry also confirmed production of minicells from both the wild type and deleted strains (see FIGS. 3A-3D).

### Example 3

Expressing Recombinant Proteins in the Cytoplasm and on the Surface of Minicells Made from GR E. coli

Minicells made from GR E. coli have many uses, including but not limited to production of minicells encapsulating biotechnologically useful proteins either in their cytoplasm or on their surfaces. To show that minicells made from GR bacteria can contain recombinant proteins within their cytoplasm, the minC minD mutants of the wild type (ME 5000) and the most highly genome reduced strain of E. coli in the TMUG collection (ME 5125; Hashimoto et al., 2005; Kato & Hashimoto, 2007) were transformed with a GFP expressing plasmid, pMP2463 (Stuurman et al., 2000). The undeleted ME 5000 and 29.7% deleted ME 5125 stains with minC minD mutations were also transformed with pRAIDA2, the plasmid that expresses an HA immunotag via an AIDA-I autotransporter surface expression cassette under the control of a rhamnose-inducible promoter (Maeda et al., 2021).

To determine the relative amounts of recombinant protein in the minicells and parental cells, immunoblots were conducted on protein extracts made from the minicells and from the parental cells used to produce the minicells, interrogating the immunoblots with antibodies directed against either GFP expressed in the cytoplasm or the HA immunotag expressed on the surface of the cells, normalizing the signal attributable to the recombinant protein to a standard, the bacterial chaperone DnaK.

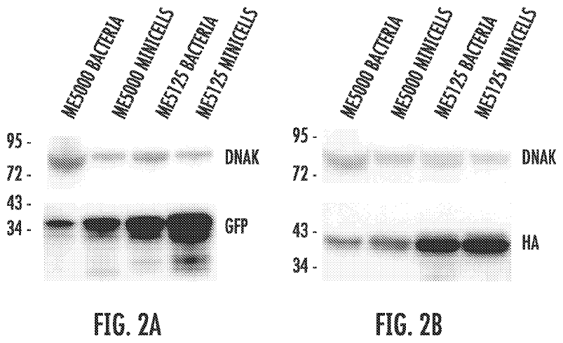

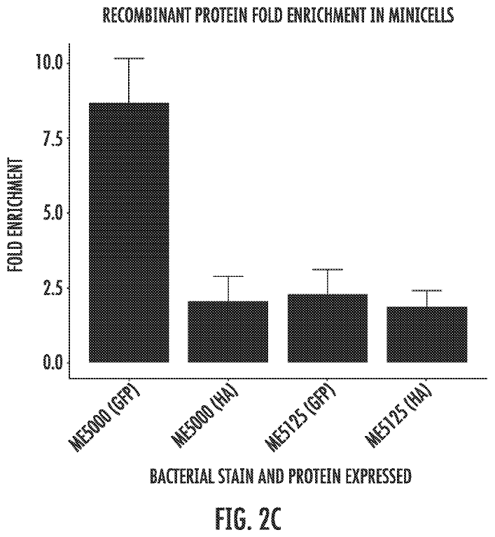

FIGS. 2A-2C show GFP and HA expression in the wild type and minC minD mutant ME 5000 and genome reduced ME 5125 E. coli strains as assessed by the immunoblots using antibodies against GFP and HA. It was determined that the GFP was present in both the parental cells and in minicells (FIG. 2A), and that the normalized amount of GFP was enriched 8.7-fold in the minicells made from ME 5000, 2.3-fold in the minicells made from ME 5125 (FIG. 2C). It was also determined that AIDA-I autotransporter-expressed HA immunotag was present in protein extracts made from the parental cells and the minicells (FIGS. 2B and 2C). The HA immunotag placed into the minicells also appeared to be concentrated in the minicells. HA was found to be present in the minicells and was enriched 2.0 fold in the minicells made from ME 5000 and 1.9-fold in the ME 5125 genome reduced bacteria. To analyze the data from the immunoblot experiment, the normalized densitometric data was log-transformed and compared the expression of GFP or HA in the minicells with the parental cells. The enrichments were statistically significant for the ME 5000 and ME 5125 cells expressing GFP or HA (p<0.01), by ANOVA analysis of the independently conducted immunoblot experiments.

### Example 4

### Image Cytometry Analysis of Non-GR and GR Bacteria Expressing GFP and HA

To further evaluate the expression of cytoplasmic- and surface-expressed recombinant proteins in non-genome reduced and genome reduced bacteria with minC minD mutations, a series of image cytometry studies were conducted. In bacteria with minC minD mutations, the cell division septum was not properly located in the middle of the cell about to undergo cell division. Instead, the cell division septum tended to form toward one pole of the bacterium, so that when cell division began to occur a small bud formed toward a pole of the parental cell, which subsequently became a minicell after the aberrant cell division process was completed. The bacteria were imaged, and images were selected that showed bacteria with and without a budding phenotype (FIGS. 3A-3D), indicating incipient production of a minicell. The images from the highly-deleted ME 5125 strain are shown. The images show that GFP was expressed in the cytoplasm and that the GFP was also present in buds at the poles of the bacteria. The images further show that HA tended to be concentrated at the poles of the bacteria, as had been described previously for wild type E. coli (Jain et al., 2006). Some of the polar structures from the parent cell appeared to show a strong concentration of the AIDA-I autotransporter surface-expressed recombinant HA in the buds.

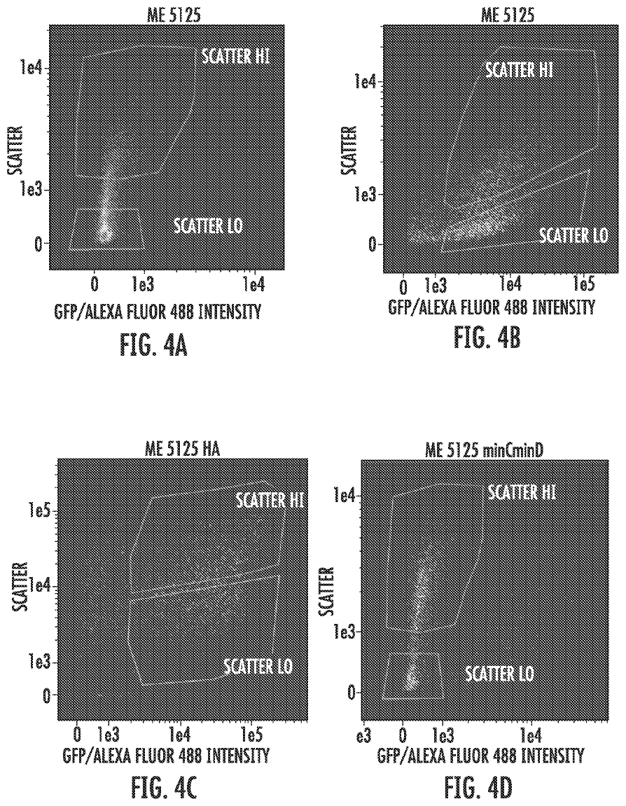

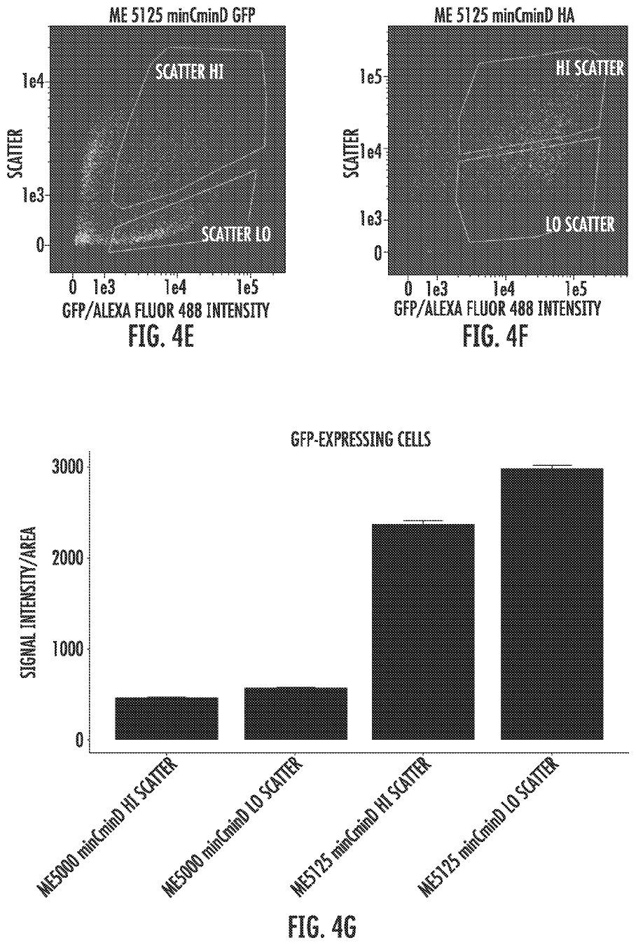

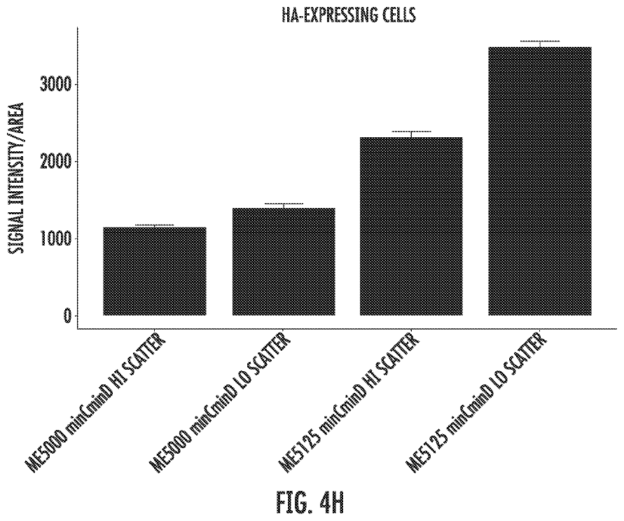

To provide another quantitative evaluation of the distribution of the GFP and HA recombinant proteins in the minicells produced from the bacteria, an analysis of the minicells identified in the image cytometry studies was conducted. FIGS. 4A-4H show a selection of the image cytometry dot plots, with the gating strategies illustrated (FIGS. 4A-4F). The signal intensity of the recombinant protein, either GFP or HA, was analyzed per unit area of each identified particle as a metric of the amount of recombinant protein in the cells and their derived minicells. Elongated structures scatter more light than spherical structures, so particles gated as low scatter preferentially represented minicells, while the particles gated as high scatter preferentially represented the whole rod-shaped E. coli. The minC minD mutations blocked the proper placement of the cell division septum so, in addition to the minicells, cells mutant in minC minD produced elongated cells that retained the bacterial chromosomes, and this elongated phenotype can be appreciated in the images of FIGS. 3A-3D. More particles gated as high scatter can be appreciated in the minC minD mutants (FIGS. 4D, 4E, and 4F). FIGS. 4G and 4H also show that the GFP and HA recombinant proteins were concentrated in the minicells (compare for both the GFP-expressing cells, FIG. 4G, and HA-expressing cells, FIG. 4H, the signal intensity/area for ME 5125 Hi Scatter vs ME 5125 Lo Scatter). The mean ratio of low scatter (minicells) to high scatter (parental cells) was 1.2 for the GFP-expressing ME 5000 cells, 1.3 for the GFP-expressing ME 5125 cells, 1.2 for the HA-expressing ME 5000 cells, and 1.5 for the HA-expressing ME 5125 cells. The differences between the Signal Intensity/Area values between low and high scatter populations for both the GFP- and HA-expressing ME 5000 and ME 5125 cells was significant (p<0.001, two-tailed t test).

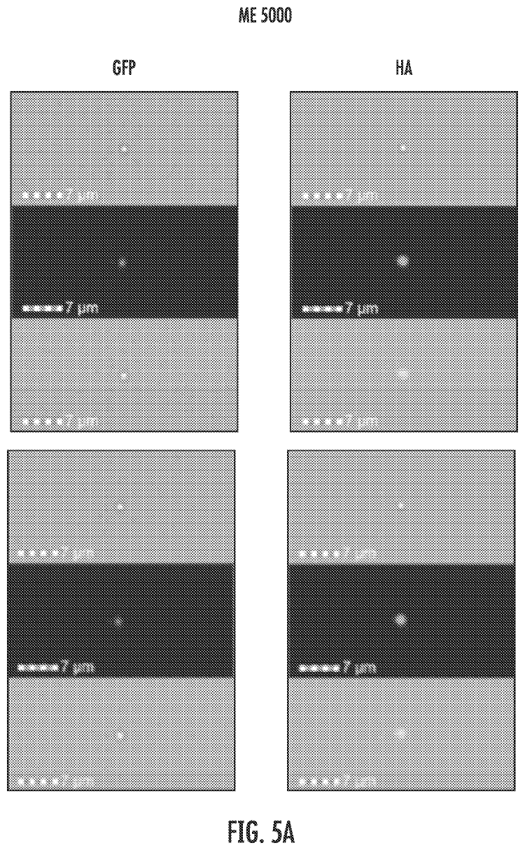

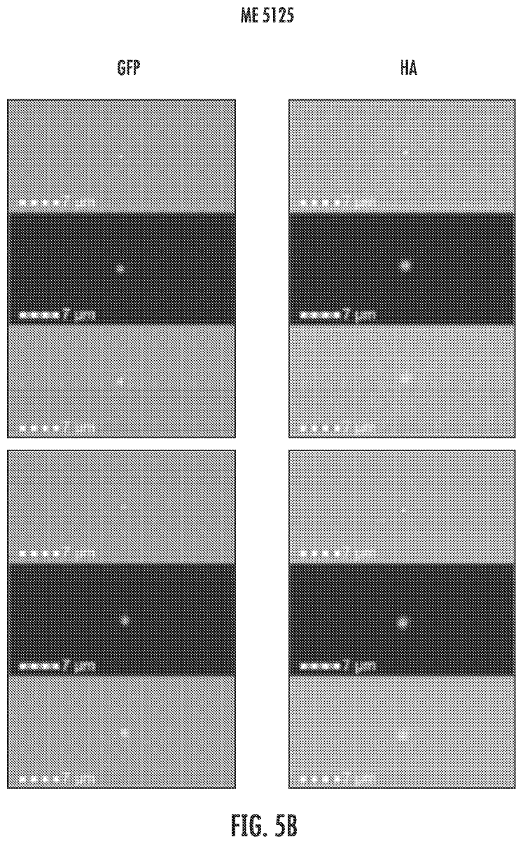

To confirm that the recombinant GFP protein and the HA immunotag expressed via the AIDA-I autotransporter are present in the minicells isolated from the minC minD mutants of the undeleted ME 5000 strain and the 29.7% genome-reduced ME 5125 strain, additional flow cytometry experiments were conducted on minicells isolated from the bacteria using a differential centrifugation procedure. FIGS. 5A and 5B show a selection of minicells isolated from the ME 5000 (FIG. 5A) and ME 5125 (FIG. 5B) strains expressing GFP or HA, demonstrating that the recombinant proteins were present in the minicells. In all cases, all minicells exhibited enhanced expression of GFP or HA.

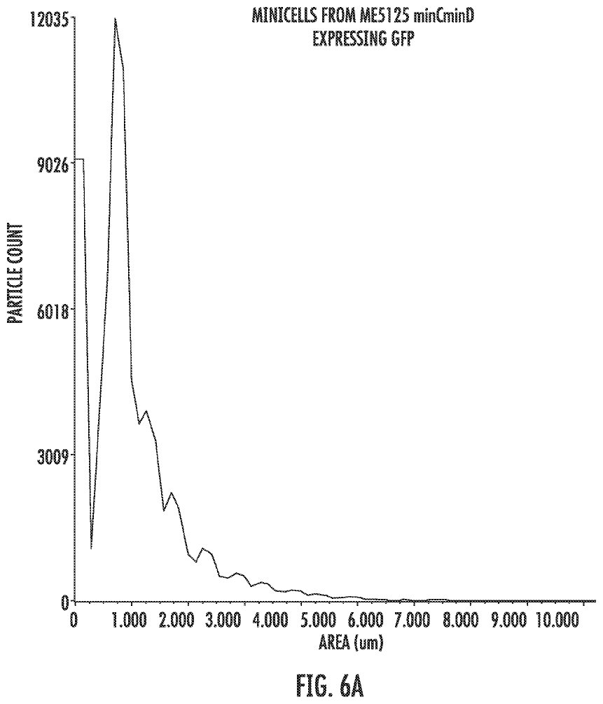

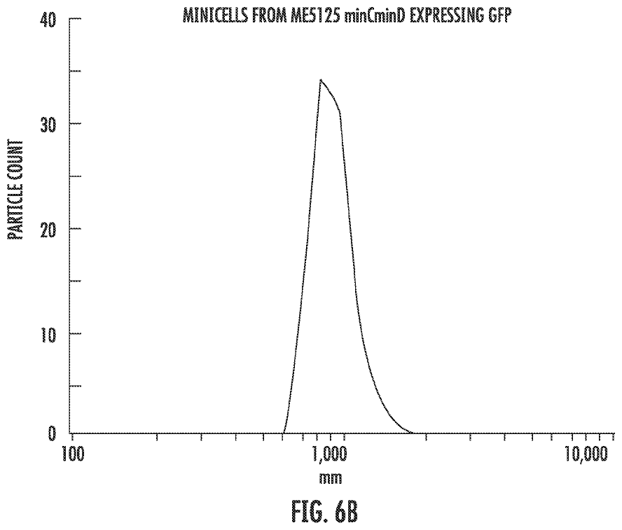

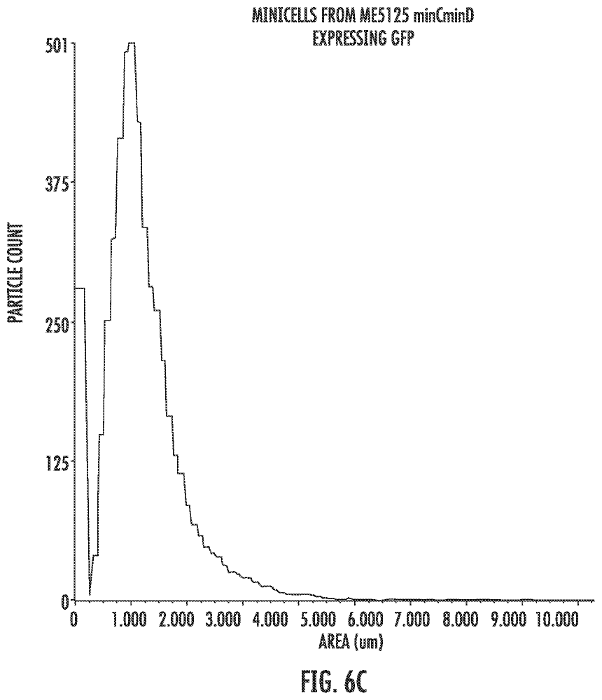

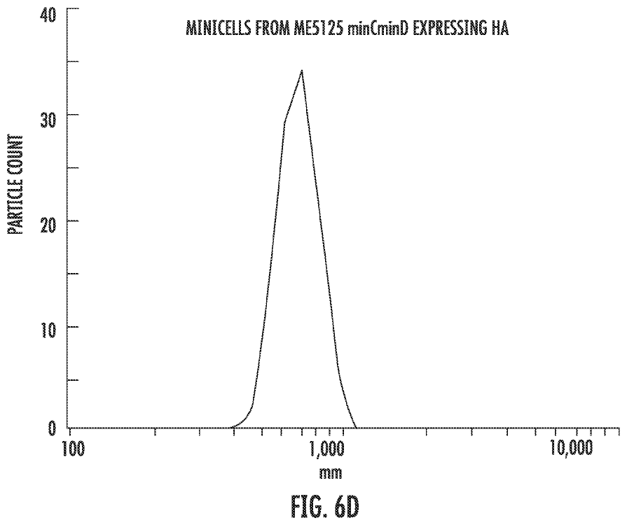

In addition to the minicells made by the minC minD mutant cells, various sorts of membrane-limited derivatives can be produced by Gram-negative bacteria including extracellular vesicles. Minicells and vesicles have distinct size distributions, with minicells having diameters of about 800 nm, while vesicles are smaller, with a diameter of about 100 nm, and certainly <250 nm (Furuyama & Sircili, 2021). To determine whether the particles that were isolated from the minC minD mutant bacteria had a size consistent with minicells, the sizes of the isolated minicells were measured using both image quantitation from the Amnis ImageStream images and by another, distinct technique, Dynamic Light Scattering (DLS) (FIGS. 6A-6D). Both approaches showed that the isolated minicells had a diameter of about 800 nm, consistent with minicells.

### Discussion of the Examples

Disclosed herein are experiments demonstrating that mutating minC and minD in GR E. coli strains produced minicells. Moreover, recombinant proteins could be successfully expressed in the genome-reduced cells, and concentrated in, and on, minicells derived from these genome-reduced E. coli. The presently disclosed subject matter thus encompasses the use of minicells made from GR E. coli in ways useful for basic investigations, bioindustry, and biomedicine. Additional advantages of a GR minicell include improved cryo-EM structural studies due to fewer bacterial structures in the minicells as well as fewer genes that engage in metabolic activities not absolutely essential for metabolism, growth, and replication.

It may be possible that the material in budding ends of the cells GFP-expressing cells represents inclusion bodies, but this is unlikely. GFP by itself, not as a fluorescent tag for a larger fusion protein, is quite soluble and does not aggregate into inclusion bodies in bacteria under most conditions. If GFP under the growth conditions employed herein did tend to aggregate into inclusion bodies, it would be expected that such inclusion bodies would be present in many or most of the bacteria, whether they were budding minicells or not, and inclusion bodies or cytoplasmic aggregates were not observed in any of the nonbudding cells.

The enrichment of the test recombinant proteins in the minicells compared to the parental cells was about 2-fold. This level of enrichment can facilitate some applications, although perhaps not others. Likely, the usefulness of the degree of enrichment will vary from application to application. For some applications, for example, the use of minicells expressing an immunogen on their surfaces as a vaccine antigen, no enrichment might be needed for enhanced effectiveness. If the smaller size of the minicells enhances transmucosal translocation and/or interaction with cells of the immune system, minicells without any enrichment in the recombinant protein can in some embodiments exhibit improved performance compared to wild type parental cells.

Bacteria are increasingly being metabolically engineered to more efficiently produce specific, industrially useful molecules (see e.g., Wendisch et al., 2006). Unneeded bacterial metabolic pathways can divert substrates to non-useful pathways and/or produce unwanted side products. If minicells are used to package recombinant proteins or metabolic products made in engineered bacteria, using minC minD strains of genome reduced, engineered bacteria can be helpful.

Expressing recombinant proteins on the surfaces of bacteria using autotransporters can be a convenient way to express and purify proteins with limited solubility. Since translation and export to the bacterial surface is very tightly coupled, recombinant low solubility proteins are placed on the surface very soon after translation so they do not have an opportunity to form insoluble aggregates or inclusion bodies within the bacterial cell (Jose & Meyer, 2007; van Ulsen et al., 2018; Meuskens et al., 2019). Expressing recombinant proteins in GR bacteria, followed by concentration in, or on, minicells may make this easier.

That both the cytoplasmically expressed protein GFP and AIDA-I autotransporter-mediated HA immunotag were concentrated in the minicells is shown herein. It may be less obvious why the cytoplasmically expressed GFP was concentrated in the minicells. However, in this case it may be helpful to consider that while the bacterial cytoplasm is sometimes thought of as being homogeneous, in fact it is not. The bacterial chromosome, and its associated proteins and structures, occupy a volume within the bacteria, so minicell cytoplasm is essentially derived from chromosome-free volume of the bacterial cytoplasm, where GFP may be present in higher abundance, resulting in selective partitioning of GFP already made in the cytoplasm into the minicells. An alternative mechanism could be the selective partitioning of the GFP-expressing plasmid into the minicells with continued protein synthesis. Minicells may therefore be a way to enhance the relative amounts of bacterial non-chromosome-associated cytoplasmic proteins for bioindustrial processes.

Minicells can also be produced from Gram-positive organisms, such as B. subtilis (Reeve et al., 1973). As such, in some embodiments it can prove biotechnologically helpful to employ highly genome-reduced and/or min mutation-bearing Gram-positive organisms for some applications (Reuß et al., 2017), and thus producing minicells from those highly genome reduced and/or min mutation-bearing Gram-positive bacteria can provide additional advantages as compared to their non-genome reduced and/or non-min mutation-bearing counterparts.

One of the important uses proposed for minicells has been as antigen delivery vehicles for new vaccines (Giacalone et al., 2006; Charlotte, 2013). No clinically approved vaccines have yet been produced using minicells, but it is plausible to hypothesize that a minicell with a surface-expressed vaccine antigen may be a more effective immunogen than a recombinant bacteria expressing the same antigen on its surface. An antigen expressed on the bacterial surface using a Gram-negative autotransporter would be more concentrated on the minicell. Minicells made from genome reduced bacteria would also include fewer host cell proteins. Minicells, because of their size and shape, may also transit across mucosal epithelial barriers more effectively and have more favorable interactions with immune system cells and so be useful for vaccine applications. We recently showed that we can place vaccine antigens on the surfaces of the same genome-reduced bacteria used in this study. We demonstrated that translation and placement on the bacterial cell surface was very tightly coupled, with minimal to no recombinant protein remaining present in the cytoplasm (Maeda et al., 2021). In that study, recombinant protein antigens derived from the fusion peptides of either SARS-COV-2 or porcine epidemic diarrhea virus (PEDV) were expressed. We showed that killed whole cell E. coli vaccines that expressed the fusion peptide antigens can elicit good anamnestic responses and protect against disease in pigs in an experimental PEDV infection model. However, this approach did not elicit a strong neutralizing antibody response (Maeda et al., 2021). An analogous minicell vaccine with an enhanced amount of antigen could yield improved immune responses. If GR bacterial surface-expressed antigen minicell vaccines prove to have enhanced immunogenicity, such vaccines may prove useful globally.

In summary, disclosed herein are minicells that can be produced from highly GR E. coli. Also disclosed is the discovery that recombinant proteins are concentrated about 2-fold in the cytoplasm and on the surfaces of the minicells. Such minicells from GR bacteria are useful for a variety of biotechnological applications.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicant reserves the right to challenge the accuracy and pertinence of any cited reference.
Adler et al. (1967) Miniature Escherichia coli cells deficient in DNA. Proc Natl Acad Sci USA 57(2):321-6.
Aguilar Suárez et al. (2019) Less Is More: Toward a Genome-Reduced Bacillus Cell Factory for “Difficult Proteins”. ACS Synth Biol 8(1): 99-108.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Anthony (2003) Transformation of Competent Bacterial Cells: Electroporation eLS. https://doi.org/10.1038/npg.els.0003749.
Barrick et al. (2017) FLP Recombination in E. coli: barricklab.org; https://barricklab.org/twiki/bin/view/Lab/ProcedureFLPFRTRecombination.
Benz & Schmidt (1989) Cloning and expression of an adhesin (AIDA-I) involved in diffuse adherence of enteropathogenic Escherichia coli. Infect Immun 57(5): 1506-11.
Benz & Schmidt (2011) Structures and functions of autotransporter proteins in microbial pathogens. International Journal of Medical Microbiology 301(6):461-8.
Bodanszky & Bodanszky (1984) in The Practice of Peptide Synthesis, Springer-Verlag, New York, New York.
Carleton et al. (2013) Engineering the type III secretion system in non-replicating bacterial minicells for antigen delivery. Nat Commun 4:1590.
Charlotte (2013) Bacterial minicells could offer safer vaccines. Nature Reviews Drug Discovery 12(5):346.
Chou & Fasman (1974) Prediction of protein conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
Colligan et al. (eds.) (1994-2004) Current Protocols in Immunology, John Wiley & Sons, Inc., New York, New York.
Daleke-Schermerhorn et al. (2014) Decoration of outer membrane vesicles with multiple antigens by using an autotransporter approach. Applied and Environmental Microbiology 80(18):5854-65.
Datsenko & Wanner (2000) One-step inactivation of chromosomal genes in Escherichia coli K-12 using PCR products. Proc Natl Acad Sci USA 97(12):6640-5.
Deutscher et al. (ed.) (1990) Guide to Protein Purification, Harcourt Brace Jovanovich, San Diego, California, United States of America.
Devereux et al. (1984) A comprehensive set of sequence analysis programs for the VAX. Nucl Acids Res 12:387-95.
Doublet et al. (2008) Antibiotic marker modifications of lambda Red and FLP helper plasmids, pKD46 and pCP20, for inactivation of chromosomal genes using PCR products in multidrug-resistant strains. J Microbiol Methods 75(2):359-61.
Farley et al. (2016) Minicells, Back in Fashion. J Bacteriol 198(8): 1186-95.
Furuyama & Sircili (2021) Outer Membrane Vesicles (OMVs) Produced by Gram-Negative
Bacteria: Structure, Functions, Biogenesis, and Vaccine Application. BioMed Res Int 2021:1490732.
GENBANK® Accession Number MW383928.1.
Gerhardt et al. (eds.) (1994) Methods for General and Molecular Bacteriology, American Society for Microbiology, Washington, DC, p. 574.
Giacalone et al. (2006) Immune responses elicited by bacterial minicells capable of simultaneous DNA and protein antigen delivery. Vaccine 24(33-34):6009-17.
Green & Sambrook (2012) Molecular Cloning: A Laboratory Manual. 4th ed. Cold Spring Harbor Laboratory Press, Cold Spring Harbor, New York, United States of America.
Gross & Mienhofer (eds.) (1981) The Peptides, Vol. 3, Academic Press, New York, New York, pp. 3-88.
Hashimoto et al. (2005) Cell size and nucleoid organization of engineered Escherichia coli cells with a reduced genome. Mol Microbiol 55(1): 137-49.
Hu et al. (2013) The Bacteriophage T7 Virion Undergoes Extensive Structural Remodeling During Infection. Science 339(6119): 576-9.
Hu et al. (2015) Structural remodeling of bacteriophage T4 and host membranes during infection initiation. Proc Natl Acad Sci USA 112(35):E4919-E28.
Ieva & Bernstein (2009) Interaction of an autotransporter passenger domain with BamA during its translocation across the bacterial outer membrane. Proc Natl Acad Sci USA 106(45): 19120-5.
Jain et al. (2006) Polar localization of the autotransporter family of large bacterial virulence proteins. J Bacteriol 188(13):4841-50.
Jong et al. (2014) An autotransporter display platform for the development of multivalent recombinant bacterial vector vaccines. Microb Cell Fact 13:162.
Jose & Meyer (2007) The autodisplay story, from discovery to biotechnical and biomedical applications. Microbiol Mol Biol Rev 71(4):600-19.
Juhas (2016) On the road to synthetic life: the minimal cell and genome-scale engineering. Crit Rev Biotechnol 36(3):416-23.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kato & Hashimoto (2007) Construction of consecutive deletions of the Escherichia coli chromosome. Mol Syst Biol 3:132.
Kaval et al. (2014) A function of DivIVA in Listeria monocytogenes division site selection. Mol Microbiol 94(3): 637-54.
Kittlesen et al. (1998) Human melanoma patients recognize an HLA-A1-restricted CTL epitope from tyrosinase containing two cysteine residues: implications for tumor vaccine development. J Immunol 60:2099-2106.
Kuipers et al. (2015) Salmonella outer membrane vesicles displaying high densities of pneumococcal antigen at the surface offer protection against colonization. Vaccine 33(17):2022-9.
Kyte & Doolittle (1982) A simple method for displaying the hydropathic character of a protein. J Mol Biol 157:105-132.
Lee et al. (2015) Generation of Minicells from an Endotoxin-Free Gram-Positive Strain Corynebacterium glutamicum. J Microbiol Biotechnol 25(4):554-8.
MacDiarmid et al. (2007) Bacterially derived 400 nm particles for encapsulation and cancer cell targeting of chemotherapeutics. Cancer Cell 11(5):431-45.
Macnab (2003) How Bacteria Assemble Flagella. Annu Rev Microbiol 57(1):77-100.
Maeda et al. (2021) Killed whole-genome reduced-bacteria surface-expressed coronavirus fusion peptide vaccines protect against disease in a porcine model. Proc Natl Acad Sci USA 118(18):e2025622118.
Meuskens et al. (2019) Type V Secretion Systems: An Overview of Passenger Domain Functions. Front Microbiol 10:1163.
Morgan & Gainor (1989) Chapter 26. Approaches to the Discovery of Non-Peptide Ligands for Peptide Receptors and Peptidases. Ann Rep Med Chem 24:243-252.
Odevall et al. (2018) The Euvichol story-Development and licensure of a safe, effective and affordable oral cholera vaccine through global public private partnerships. Vaccine 36(45):6606-14.
PCT International Patent Application Publication Nos. WO 1999/54464; WO 2004/110486; WO 2006/012539; WO 2006/012539; WO 2020/046982; WO 2021/076897; WO 2021/231441.
PCT International Patent Application Serial No. PCT/US2005/026102.
Rapp & Kaufmann (2004) DNA vaccination with gp96-peptide fusion proteins induces protection against an intracellular bacterial pathogen. Int Immunol 16:597-605.
Remington's Pharmaceutical Sciences, 18th ed. (1990) Gennaro (ed.) Mack Publishing, Easton, Pennsylvania, United States of America.
Reeve et al. (1973) Minicells of Bacillus subtilis. J Bacteriol 114(2):860-873.
Reuß et al. (2017) Large-scale reduction of the Bacillus subtilis genome: consequences for the transcriptional network, resource allocation, and metabolism. Genome Res 27(2):289-299.
Schraidt & Marlovits (2011) Three-Dimensional Model of Salmonella's Needle Complex at Subnanometer Resolution. Science 331(6021): 1192-5.
Shepherd et al. (2001) Cytoplasmic RNA Polymerase in Escherichia coli. J Bacteriol 183(8):2527-34.
Spatola (1983) Chemistry and Biochemistry of Amino Acids, Peptides and Proteins, Vol. VII. Weinstein (ed.). Marcel Dekker, Inc., New York, New York, pp. 267-357.
Stewart et al. (1984) in Solid Phase Peptide Synthesis, 2nd Edition, Pierce Chemical Company, Rockford, Illinois.
Stuurman et al. (2000) Use of green fluorescent protein color variants expressed on stable broad-host-range vectors to visualize rhizobia interacting with plants. Mol Plant Microbe Interact 13(11):1163-9.
Sun et al. (2014) Icosahedral bacteriophage ΦX174 forms a tail for DNA transport during infection. Nature 505(7483):432-5.
Sung et al. (2016) Construction of a minimal genome as a chassis for synthetic biology. Essays in Biochemistry 60(4):337-46.
U.S. Patent Application Publication No. 2008/0193469.
U.S. patent application Ser. No. 11/572,453.
van Tilburg et al. (2020) MiniBacillus PG10 as a Convenient and Effective Production Host for Lantibiotics. ACS Synth Biol 9(7): 1833-1842.
van Ulsen et al. (2018) On display: autotransporter secretion and application. FEMS Microbiol Letters 365(18):fny165.
Ward Jr. & Lutkenhaus (1985) Overproduction of FtsZ induces minicell formation in E. coli. Cell 42(3):941-9.
Welters et al. (2004) Chemically synthesized protein as tumour-specific vaccine: immunogenicity and efficacy of synthetic HPV16 E7 in the TC-1 mouse tumour model. Vaccine 23:305-311.
Wendisch et al. (2006) Metabolic engineering of Escherichia coli and Corynebacterium glutamicum for biotechnological production of organic acids and amino acids. Current Opinion in Microbiology 9(3): 268-74.
Zhang et al. (2010) How to make a minimal genome for synthetic minimal cell. Protein Cell 1(5):427-34.
Zugel (2001) gp96-peptide vaccination of mice against intracellular bacteria Infect Immun 69:4164-4167.

It will be understood that various details of the presently disclosed subject matter can be changed without departing from the scope of the presently disclosed subject matter. Furthermore, the foregoing description is for the purpose of illustration only, and not for the purpose of limitation.

1. A bacterial minicell derived from a bacterium, wherein the bacterium is a genome reduced (GR) bacterium having a reduced number of expressed genes and/or is a bacterium having one or more mutated min genes and/or is a GR bacterium having one or more mutated min genes.

2. The bacterial minicell of claim 1, wherein the one or more mutated min genes are a minC gene and/or a minD gene.

3. The bacterial minicell of claim 1, wherein the minicell comprises a recombinant protein.

4. The bacterial minicell of claim 3, wherein the recombinant protein is present in cytoplasm and/or on a surface of the minicell.

5. The bacterial minicell of claim 3, wherein the recombinant protein is present in the minicell in an enhanced amount as compared to an amount that would have been present in a minicell derived from a bacterium of the same strain that has a full complement of expressed genes and/or an unmutated min gene.

6. The bacterial minicell of claim 3, wherein the recombinant protein is an antigen, optionally an antigen on a surface of a membrane, or a derivative thereof.

7. The bacterial minicell of claim 6, wherein the minicell induces an enhanced immune response against the antigen when administered to a subject as compared to an immune response that would have been induced in the subject by a minicell derived from a bacterium of the same strain that has a full complement of expressed genes and/or an unmutated min gene.

8. The bacterial minicell of claim 7, wherein reducing and/or eliminating expression of one or more genes and/or mutating the one or more min genes in the bacterium yields enhanced immune response.

9. The bacterial minicell of claim 1, wherein the bacterium is a Gram-negative bacterium, optionally a member of the Enterobacteriaceae.

10. The bacterial minicell of claim 1, wherein the bacterium is an E. coli, an a Shigella bacterium, a Yersinia bacterium, or a Salmonella bacterium.

11. The bacterial minicell of claim 1, wherein the reduced number of expressed genes comprises a reduction of at least about 1.0%, 2.0%, 3.0%, 4.0%, 5.0%, 6.0%, 7.0%, 8.0%, 9.0%, 10%, 11%, 12%, 13%, 14%, 15%, or greater than 15% of genes.

12. The bacterial minicell of claim 11, wherein the reduced number of expressed genes comprises a reduction of expressed genes selected from the group consisting of at least about 2.4%, at least about 15.9%, and at least about 29.7%.

13. The bacterial minicell of claim 1, wherein the recombinant protein, optionally the antigen, is put on the surface of the minicell by an approach selected from the group consisting of expression by the bacterium, covalent or non-covalent association with the outer membrane, and combinations thereof.

14. The bacterial minicell of claim 13, wherein the bacterium comprises an autotransporter (AT) expression vector encoding the recombinant protein, optionally the antigen, wherein the expression on the surface is provided by the AT expression vector.

15. The bacterial minicell of claim 14, wherein the autotransporter expression vector comprises a codon optimized sequence encoding the antigen.

16. The bacterial minicell of claim 14, wherein the AT expression vector comprises a monomeric autotransporter vector or a trimeric autotransporter vector.

17. The bacterial minicell of claim 3, wherein the recombinant protein, optionally the antigen, is derived from a microbe.

18. The bacterial minicell of claim 3, wherein the recombinant protein, optionally the antigen, is derived from a tumor and/or a cancer, a target of an inappropriate or undesirable immune response, or a component of the host immune system, optionally a host immune system component that, when targeted for destruction, inactivation, or activation, alters an undesirable immune response.

19. A method for producing an antibody in a subject, the method comprising providing a minicell according to claim 6 and administering the minicell to a subject in an amount and via a route sufficient to produce an antibody in the subject against the antigen present in or on the minicell, optionally wherein the production of the antibody is enhanced in the subject as compared to that which would have been induced in the subject by a minicell derived from a bacterium of the same strain that has a full complement of expressed genes and/or an unmutated min gene.

20. The method of claim 19, comprising administering the minicell to the subject intranasally, transmucosally, including but not limited to orally, rectally, and vaginally; subcutaneously, intradermally, intramuscularly, other parenteral routes, or any combination thereof.

21. A vaccine composition comprising a bacterial minicell according to claim 6 and a pharmaceutically acceptable carrier, optionally wherein the vaccine composition further comprises one or more adjuvants.

22. The vaccine composition of claim 21, wherein the vaccine composition is adapted to be administered orally, rectally, vaginally, intra-nasally, parenterally, intradermally, subcutaneously, or intramuscularly.

23. The vaccine compostions of claim 21, wherein the vaccine composition further comprises an adjuvant.

24. A method for vaccinating a subject in need thereof, the method comprising providing a vaccine composition according to claim 21 and administering the vaccine composition to the subject.

25. A method for treating a cancer or inappropriate immune responses or expression or production of a deleterious material in a subject in need thereof, the method comprising providing a vaccine composition according to claim 21 and administering the vaccine to the subject.

26. The method of claim 24, wherein the vaccine composition is administered orally, rectally, vaginally, intra-nasally, parenterally, intradermally, subcutaneously, or intramuscularly.

27. An expression vector comprising a nucleotide sequence encoding a heterologous protein, optionally an antigen, wherein the expression vector is configured to express the heterologous protein in and/or on the surface of a minicell derived from a modified bacterium having a reduced number of expressed genes and/or one or more mutated min genes.

28. The expression vector of claim 27, comprising an autotransporter (AT) expression vector.

29. The expression vector of claim 27, wherein the vector comprises a codon optimized sequence encoding the antigen.

30. The expression vector of claim 27, wherein the AT expression vector comprises a monomeric vector or a trimeric vector.

31. The expression vector of claim 27, wherein the nucleotide sequence encoding the heterologous protein, optionally an antigen, is positioned under control of an inducible promoter or a constitutive promoter.

32. The expression vector of claim 27, wherein the heterologous protein, optionally an antigen, is expressed as a monomer or as a trimer.

33. The expression vector of claim 27, provided in a pharmaceutically acceptable carrier.
